SEQUENCE LISTING (4 sequences)

SEQ ID NO: 1 - DNA, 8043 bp, H. sapiens
  misc_feature (7568)...(7568): n = A, T, C or G
cccgccccgacgccgcgtccctgcagccctgcccggcgctccagtagcaggacccggtctcgggaccagccggtaatatgcacgtgtcactagctgaggccctggaggttcggggtggaccacttcaggaggaagaaatatgggctgtattaaatcaaagtgctgaaagtctccaagaattattcagaaaagtaagcctagctgatcctgctgcccttggcttcatcatttctccatggtctctgctgttgctgccatctggtagtgtgtcatttacagatgaaaatatttccaatcaggatcttcgagcattcactgcaccagaggttcttcaaaatcagtcactaacttctctctcagatgttgaaaagatccacatttattctcttggaatgacactgtattggggggctgattatgaagtgcctcagagccaacctattaagcttggagatcatctcaacagcatactgcttggaatgtgtgaggatgttatttacgctcgagtttctgttcggactgtgctggatgcttgcagtgcccacattaggaatagcaattgtgcaccctcattttcctacgtgaaacacttggtaaaactggttctgggaaatctttctgggacagatcagctttcctgtaacagtgaacaaaagcctgatcgaagccaggctattcgagatcgattgcgaggaaaaggattaccaacaggaagaagctctacttctgatgtactagacatacaaaagcctccactctctcatcagacctttcttaacaaagggcttagtaaatctatgggatttctgtccatcaaagatacacaagatgagaattatttcaaggacattttatcagataattctggacgtgaagattctgaaaatacattctccccttaccagttcaaaactagtggcccagaaaaaaaacccatccctggcattgatgtgctttctaagaagaagatctgggcttcatccatggacttgctttgtacagctgacagagacttctcttcaggagagactgccacatatcgtcgttgtcaccctgaggcagtaacagtgcggacttcaactacgcctagaaaaaaggaggcaagatactcagatggaagtatagccttggatatctttggccctcagaaaatggatccaatatatcacactcgagaattgcccacctcctcagcaatatcaagtgctttggaccgaatccgagagagacaaaagaaacttcaggttctgagggaagccatgaatgtagaagaaccagttcgaagatacaaaacttatcatggtgatgtctttagtacctccagtgaaagtccatctattatttcctctgaatcagatttcagacaagtgagaagaagtgaagcctcaaagaggtttgaatccagcagtggtctcccaggggtagatgaaaccttaagtcaaggccagtcacagagaccgagcagacaatatgaaacaccctttgaaggcaacttaattaatcaagagatcatgctaaaacggcaagaggaagaactgatgcagctacaagccaaaatggcccttagacagtctcggttgagcctatatccaggagacacaatcaaagcgtccatgcttgacatcaccagggatccgttaagagaaattgccctagaaacagccatgactcaaagaaaactgaggaatttctttggccctgagtttgtgaaaatgacaattgaaccatttatatctttggatttgccacggtctattcttactaagaaagggaagaatgaggataaccgaaggaaagtaaacataatgcttctgaacgggcaaagactggaactgacctgtgataccaaaactatatgtaaagatgtgtttgatatggttgtggcacatattggcttagtagagcatcatttgtttgctttagctaccctcaaagataatgaatatttctttgttgatcctgacttaaaattaaccaaagtggccccagagggatggaaagaagaaccaaagaaaaagaccaaagccactgttaattttactttgtttttcagaattaaattttttatggatgatgttagtctaatacaacatactctgacgtgtcatcagtattaccttcagcttcgaaaagatattttggaggaaaggatgcactgtgatgatgagacttccttattgctggcatccttggctctccaggctgagtatggagattatcaaccagaggttcatggtgtgtcttactttagaatggagcactatttgcccgccagagtgatggagaaacttgatttatcctatatcaaagaagagttacccaaattgcataatacctatgtgggagcttctgaaaaagagacagagttagaatttttaaaggtctgccaaagactgacagaatatggagttcattttcaccgagtgcaccctgagaagaagtcacaaacaggaatattgcttggagtctgttctaaaggtgtccttgtgtttgaagttcacaatggagtgcgcacattggtccttcgctttccatggagggaaaccaagaaaatatctttttctaaaaagaaaatcacattgcaaaatacatcagatggaataaaacatggcttccagacagacaacagtaagatatgccagtacctgctgcacctctgctcttaccagcataagttccagctacagatgagagcaagacagagcaaccaagatgcccaagatattgagagagcttcgtttaggagcctgaatctccaagcagagtctgttagaggatttaatatgggacgagcaatcagcactggcagtctggccagcagcaccctcaacaaacttgctgttcgacctttatcagttcaagctgagattctgaagaggctatcctgctcagagctgtcgctttaccagccattgcaaaacagttcaaaagagaagaatgacaaagcttcatgggaggaaaagcctagagagatgagtaaatcataccatgatctcagtcaggcctctctctatccacatcggaaaaatgtcattgttaacatggaacccccaccacaaaccgttgcagagttggtgggaaaaccttctcaccagatgtcaagatctgatgcagaatctttggcaggagtgacaaaacttaataattcaaagtctgttgcgagtttaaatagaagtcctgaaaggaggaaacatgaatcagactcctcatccattgaagaccctgggcaagcatatgttctagatgtgctacacaaaagatggagcatagtatcttcaccagaaagggagatcaccttagtgaacctgaaaaaagatgcaaagtatggcttgggatttcaaattattggtggggagaagatgggaagactggacctaggcatatttatcagctcagttgcccctggaggaccagctgacttccatggatgcttgaagccaggagaccgtttgatatctgtgaatagtgtgagtctggagggagtcagccaccatgctgcaattgaaattttgcaaaatgcacctgaagatgtgacacttgttatctctcagccaaaagaaaagatatccaaagtgccttctactcctgtgcatctcaccaatgagatgaaaaactacatgaagaaatcttcctacatgcaagacagtgctatagattcttcttccaaggatcaccactggtcacgtggtaccctgaggcacatctcggagaactcctttgggccgtctgggggcctgcgggaaggaagcctgagttctcaagattccaggactgagagtgccagcttgtctcaaagccaggtcaatggtttctttgccagccatttaggtgaccaaacctggcaggaatcacagcatggcagcccttccccatctgtaatatccaaagccaccgagaaagagactttcactgatagtaaccaaagcaaaactaaaaagccaggcatttctgatgtaactgattactcagaccgtggagattcagacatggatgaagccacttactccagcagtcaggatcatcaaacaccaaaacaggaatcttcctcttcagtgaatacatccaacaagatgaattttaaaactttttcttcatcacctcctaagcctggagatatctttgaggttgaactggctaaaaatgataacagcttggggataagtgtcacgggaggtgtgaatacgagtgtcagacatggtggcatttatgtgaaagctgttattccccagggagcagcagagtctgatggtagaattcacaaaggtgatcgcgtcctagctgtcaatggagttagtctagaaggagccacccataagcaagctgtggaaacactgagaaatacaggacaggtggttcatctgttattagaaaagggacaatctccaacatctaaagaacatgtcccggtaaccccacagtgtaccctttcagatcagaatgcccaaggtcaaggcccagaaaaagtgaagaaaacaactcaggtcaaagactacagctttgtcactgaagaaaatacatttgaggtaaaattatttaaaaatagctcaggtctaggattcagtttttctcgagaagataatcttataccggagcaaattaatgccagcatagtaagggttaaaaagctctttgctggacagccagcagcagaaagtggaaaaattgatgtaggagatgttatcttgaaagtgaatggagcctctttgaaaggactatctcagcaggaagtcatatctgctctcaggggaactgctccagaagtattcttgcttctctgcagacctccacctggtgtgctaccggaaattgatactgcgcttttgaccccacttcagtctccagcacaagtacttccaaacagcagtaaagactcttctcagccatcatgtgtggagcaaagcaccagctcagatgaaaatgaaatgtcagacaaaagcaaaaaacagtgcaagtccccatccagaagagacagttacagtgacagcagtgggagtggagaagatgacttagtcacagctccagcaaacatatcaaattcgacctggagttcagctttgcatcagactctaagcaacatggtatcacaggcacagagtcatcatgaagcacccaagagtcaagaagataccatttgtaccatgttttactatcctcagaaaattcccaataaaccagagtttgaggacagtaatccttcccctctaccaccggatatggctcctgggcagagttatcaaccccaatcagaatctgcttcctctagttcgatggataagtatcatatacatcacatttctgaaccaactagacaagaaaactggacacctttgaaaaatgacttggaaaatcaccttgaagactttgaactggaagtagaactcctcattaccctaattaaatcagaaaaagcaagcctgggttttacagtaaccaaaggcaatcagagaattggttgttatgttcatgatgtcatacaggatccagccaaaagtgatggaaggctaaaacctggggaccggctcataaaggttaatgatacagatgttactaatatgactcatacagatgcagttaatctgctccgggctgcatccaaaacagtcagattagttattggacgagttctagaattacccagaataccaatgttgcctcatttgctaccggacataacactaacgtgcaacaaagaggagttgggtttttccttatgtggaggtcatgacagcctttatcaagtggtatatattagtgatattaatccaaggtccgtcgcagccattgagggtaatctccagctattagatgtcatccattatgtgaacggagtcagcacacaaggaatgaccttggaggaagttaacagagcattagacatgtcacttccttcattggtattgaaagcaacaagaaatgatcttccagtggttcccagctcaaagaggtctgctgtttcagctccaaagtcaaccaaaggcaatggttcctacagtgtggggtcttgcagccagcctgccctcactcctaatgattcattctccacggttgctggggaagaaataaatgaaatatcgtaccccaaaggaaaatgttctacttatcagataaagggatcaccaaacttgactctgcccaaagaatcttatatacaagaagatgacatttatgatgattcccaagaagctgaagttatccagtctctgctggatgttgttgatgaggaagcccagaatcttttaaacgaaaataatgcagcaggatactcctgtggtccaggtacattaaagatgaatgggaagttatcagaagagagaacagaagatacagactgcgatggttcacctttacctgagtattttactgaggccaccaaaatgaatggctgtgaagaatattgtgaagaaaaagtaaaaagtgaaagcttaattcagaagccacaagaaaagaagactgatgatgatgaaataacatggggaaatgatgagttgccaatagagagaacaaaccatgaagattctgataaagatcattcctttctgacaaacgatgagctcgctgtactccctgtcgtcaaagtgcttccctctggtaaatacacgggtgccaacttaaaatcagtcattcgagtcctgcggggtttgctagatcaaggaattccttctaaggagctggagaatcttcaagaattaaaacctttggatcagtgtctaattgggcaaactaaggaaaacagaaggaagaacagatataaaaatatacttccctatgatgctacaagagtgcctcttggagatgaaggtggctatatcaatgccagcttcattaagataccagttgggaaagaagagttcgtttacattgcctgccaaggaccactgcctacaactgttggagacttctggcagatgatttgggagcaaaaatccacagtgatagccatgatgactcaagaagtagaaggagaaaaaatcaaatgccagcgctattggcccaacatcctaggcaaaacaacaatggtcagcaacagacttcgactggctcttgtgagaatgcagcagctgaagggctttgtggtgagggcaatgacccttgaagatattcagaccagagaggtgcgccatatttctcatctgaatttcactgcctggccagaccatgatacaccttctcaaccagatgatctgcttacttttatctcctacatgagacacatccacagatcaggcccaatcattacgcactgcagtgctggcattggacgttcagggaccctgatttgcatagatgtggttctgggattaatcagtcaggatcttgattttgacatctctgatttggtgcgctgcatgagactacaaagacacggaatggttcagacagaggatcaatatattttctgctatcaagtcatcctttatgtcctgacacgtcttcaagcagaagaagagcaaaaacagcagcctcagcttctgaagtgacatgaaaagagcctctggatgcatttccatttctctccttaacctccagcagactcctgctctctatccaaataaagatcacagagcagnaagttcatacaacatgcatgttctcctctatcttagaggggtattcttcttgaaaataaaaaatattgaaatgctgtatttttacagctactttaacctatgataattatttacaaaattttaacactaaccaaacaatgcagatcttagggatgattaaaggcagcattgatgatagcaagacattgttacaaggacatggtgagtctatttttaatgcaccaatcttgtttatagcaaaaatgttttccaatattttaataaagtagttattttatagggcatacttgaaaccagtatttaagctttaaatgacagtaatattggcatagaaaaaagtagcaaatgtttactgtatcaatttctaatgtttactatatagaatttcctgtaatatatttatatactttttcatgaaaatggagttatcagttatctgtttgttactgcatcatctgtttgtaatcattatctc

SEQ ID NO: 2 - protein, 2466 aa, H. sapiens [sequence omitted]

SEQ ID NO: 3 - RNA, 21 nt, Artificial Sequence
  siRNA
aaguaagccuagcugauccug

SEQ ID NO: 4 - protein, 9 aa, Artificial Sequence
  artificial substrate
KKEAXDTLI

(19) United States
(12) Patent Application Publication
(10) Pub. No.: US 2004/0224337 A1
(43) Pub. Date: Nov. 11, 2004

(54) Use of biomolecular targets in the treatment and visualization of tumors

(76) Inventors: Erik Foehr, Novato, CA (US); Jasna Jerecic, San Francisco, CA (US); Gustavo A. Lorente, Millbrae, CA (US); Roman Urfer, Belmont, CA (US)

Correspondence Address: BOZICEVIC, FIELD & FRANCIS LLP, 1900 University Ave, East Palo Altho, CA, 94303, US

(21) Appl. No.: 10/795,148

(22) Filed: Mar. 4, 2004

Related U.S. Application Data
(60) Provisional application No. 60/452,169, filed on Mar. 4, 2003.

(51) Int. Cl. C12Q 1/68; G01N 33/53; G01N 33/567; A01K 67/00

(52) U.S. Cl. 435/6; 800/9; 435/7.2

(57) ABSTRACT

PTPL1/FAP-1 is shown to be differentially expressed in primary brain tumor tissues, as compared to normal brain tissues. PTPL1/FAP-1 is useful as a biomolecular target for brain tumor treatment therapies. Agents are screened for their effect on PTPL1/FAP-1, and find use as therapeutic agents. Determination of PTPL1/FAP-1 overexpression provides diagnostic tests for detecting and staging brain tumors. The invention also provides compounds and pharmaceutically acceptable compositions for administration to patients suffering from a brain tumor.

### BACKGROUND OF THE INVENTION

Brain tumors represent a unique challenge for drug development. Because of the vital and diverse function of the different parts of the brain the most effective treatment of other cancers, surgery, is problematic. Further most brain tumors are relatively insensitive to other cancer treatments, including radiation and chemotherapy. Among the diverse group of brain tumors (the World Health Organization lists 126), astrocytic tumors (Grade I-IV) are the most common. Astrocytoma Grade IV (Glioblastoma) are the most deadly. Several grades of tumors are described based on the cell composition, mitotic index and morphological characteristics. Astrocytic tumors are very diverse and may represent a continuum of progressively more deadly cancers. There is no effective treatment for brain cancer. Use of surgery is problematic and radiation and chemotherapy have been met with limited success.

Glioblastomas are the most malignant astrocytic tumors, composed of poorly differentiated neoplastic cells. Glioblastoma typically affects adults and is preferentially located in the white matter of cerebral hemispheres. Glioblastomas may develop from low grade astrocytomas (type I glioblastoma) or more frequently they manifest de novo (type II glioblastoma). The GBMs are composed of poorly undifferentiated, often pleomorphic astrocytic cells with marked nuclear atypia and brisk mitotic activity. Prominent microvascular proliferation and/or necrosis are essential diagnostic features. GBM shows a strong regional heterogeneity, which poses a serious challenge for the analysis of these tumors. GBM is the most frequent brain tumor representing 50-60% of all astrocytic tumors and 20% of all intracranial tumors. It has a peak incidence at age 45 to 60. The mean survival time after diagnosis is less then a year. Pathologically, the diagnosis of GBM requires a heterogeneous tumor with areas of necrosis and/or prominent vascular proliferation.

The brain is encased in the rigid shell of the skull, and is cushioned by the cerebrospinal fluid. Because of the relatively small volume of the skull cavity, minor changes in the volume of tissue in the brain can dramatically increase intracranial pressure, causing damage to the entire organ. Thus, even small tumors can have a profound and adverse affect on the brain's function. The cramped physical location of the cranium also makes surgery and treatment of the brain a difficult and delicate procedure. However, because of the dangers of increased intracranial pressure from the tumor, surgery is often the first strategy of attack in treating brain tumors.

In addition to its physical isolation, the brain is chemically and biologically isolated from the rest of the body by the “Blood-Brain-Barer” (or BBB). This physiological phenomenon is due to the “tightness” of the epithelial cell junctions in the lining of the blood vessels in the brain. Nutrients, which are actively transported across the cell lining, can reach the brain, but other molecules from the bloodstream are excluded. This prevents toxins, viruses, and other potentially dangerous molecules from entering the brain cavity. However, it also prevents therapeutic molecules, including many chemotherapeutic agents that are useful in other types of tumors, from crossing into the brain. Thus, many therapies directed at the brain must be delivered directly into the brain cavity, e.g. by an Ommaya reservoir, or administered in elevated dosages to ensure the diffusion of an effective amount across the BBB.

With the difficulties of administering chemotherapies to the brain, radiotherapy approaches have also been attempted. However, the amount of radiation necessary to completely destroy potential tumor-producing cells also produces unacceptable losses of healthy brain tissue. The retention of patient cognitive function while eliminating the tumor mass is another challenge to brain tumor treatment. Neoplastic brain cells are often pervasive, and travel throughout the entire brain mass. Thus, it is impossible to define a true “tumor margin,” unlike, for example, in lung or bladder cancers. Unlike reproductive (ovarian, uterine, testicular, prostate, etc.), breast, kidney, or lung cancers, the entire organ, or even significant portions, cannot be removed to prevent the growth of new tumors. In addition, brain tumors are very heterogeneous, with different cell doubling times, treatment resistances, and other biochemical idiosyncrasies between the various cell populations that make up the tumor. This pervasive and variable nature greatly adds to the difficulty of treating brain tumors while preserving the health and function of normal brain tissue.

Although current surgical methods offer considerably better post-operative life for patients, current combination therapy methods (surgery, low-dosage radiation, and chemotherapy) have only improved the life expectancy of patients by one month, as compared to the methods of 30 years ago. Without effective agents to prevent the growth of brain tumor cells that are present outside the main tumor mass, the prognosis for these patients cannot be significantly improved. Although some immuno-affinity agents have been proposed and tested for the treatment of brain tumors, see, for example, the tenascin-targeting agents described in U.S. Pat. No. 5,624,659, these agents have not proven sufficient for the treatment of brain tumors. Thus, therapeutic agents which are directed towards new molecular targets, and are capable of specifically targeting and killing brain tumor cells, are urgently needed for the treatment of brain tumors.

Relevant Literature

Analysis of differential gene expression in glioblastoma may be found in, for example, Mariani et al. (2001) J Neurooncol 53(2):161-76; Markert et al. (2001) Physiol Genomics 5(1):21-33; Yano et al. (2000) Neurol Res 22(7):650-6; Kroes et al. (2000) Cancer Lett 156(2):191-8; and Reis et al. (2000) Am J Pathol 156(2):425-32, among others.

The sequence of PTPL1/FAP-1 may be found, inter alia, in U.S. Pat. No. 5,821,075; and U.S. Pat. No. 6,066,472. FAS associated phosphatases are disclosed in U.S. Pat. No. 5,876,939.

Saras et al. (1997) J. Biol. Chem. 272:24333-24338 describe the interaction of a GTPase-activating protein with PTPL1/FAP-1. Murthy et al. (1999) J. Biol. Chem. 274:20679-20687 discuss an interaction of ZRP-1 with PTPL1/FAP-1. The differential expression of PTPL1/FAP-1 after acute arterial injury is discussed by Wright et al. (2000) Arterioscler. Thromb. Vasc. Biol. 20:1189-1198. Hehner et al. (1999) Eur. J. Biochem. 264:132-139 suggest the role of tyrosine phosphatase in FAS mediated apoptosis.

### SUMMARY OF THE INVENTION

The present invention provides methods and reagents for specifically targeting neoplastic cells, particularly brain tumors, for both therapeutic and imaging purposes, by targeting the protein, PTPL1/FAP-1. This target is identified as being overexpressed in brain and other tumors. The selective inhibition of PTPL1/FAP-1 in tumor cells results in the cells having a greater sensitivity to programmed cell death, thereby improving the efficacy of chemotherapeutic agents. Tumor cells are also selectively marked with therapeutic or visualizing compositions that have a specific affinity for PTPL1/FAP-1. The invention also provides methods for the identification of compounds that modulate the expression of genes encoding PTPL1/FAP-1, or the activity of the PTPL1/FAP-1 gene product, as well as methods for the treatment of disease by administering such compounds to individuals suffering from such tumors.

In one embodiment of the invention, compounds useful in the treatment of tumors are assayed for an ability to inhibit the dephosphorylation of a substrate by FAP-1. Such assays may include FAP-1 or an active fragment thereof; and a substrate of FAP-1, where the substrate is phosphorylated. A substrate of interest may include a peptide derived from FAS. Inhibitors of FAP-1 prevent the specific dephosphorylation of the substrate. In other embodiments of the invention, compounds useful in the treatment of tumors are assayed by their effect on cells that express FAP-1, e.g. glioma cells. Such effects may include the ability of the cells to undergo programmed cell death; the activation of proteins involved in apoptosis, e.g. caspase 3, PARP, etc.; and the like.

### BRIEF DESCRIPTION OF THE DRAWINGS

FIG. 1. PTPL1/FAP-1 gene expression is upregulated in glioblastoma multiforme. Quantitative PCR was performed on four normal human brain samples and compared to three glioblastoma tumor samples as well as other brain tumor sub-types. The amount of PTPL1/FAP-1 mRNA was calculated relative to the “house keeping” β-Actin. In all three glioblastoma samples PTPL1/FAP-1 was upregulated when compared to normal brain samples.

FIG. 2. PTPL1/FAP-1 expression in human GBM tissue. Immunohistochemical staining for PTPL1/FAP-1 in GBM tissue (Brown Stain) indicates' that PTPL1/FAP-1 protein is expressed.

FIG. 3A-B. a) PTPL1/FAP-1 siRNA inhibits glioma cell growth. Human glioma tumor cells (D566) were transfected with Scrambled siRNA (negative control), PTPL1/FAP-1 siRNA, (test), mock transfected (no siRNA), or were not transfected (non-treated). The cells were then monitored for 3 days to measure cell proliferation. (b) PTPL1/FAP-1 protein levels were measured by immunoblotting with PTPL1/FAP-1 antibody in the treated lysates at the times indicated (upper panel). The blots were subsequently reprobed with β-actin as a loading control (lower panel).

FIG. 4. a) PTPL1/FAP-1 siRNA increases FAS induced apoptosis. Human glioma tumor cells (D566) were transfected with Scrambled siRNA (negative control), or PTPL1/FAP-1 siRNA. The cells were then treated with recombinant FAS ligand for 18 hours and then cell viability was measured.

FIG. 5. FAP-1 expression in cell lines. Fifty μg of protein extract from different cell lines was immunoblotted for FAP-1: (1) Jurkat (2) HEK293, (3) HeLa (4) SKOV-3 (5) PANC-1, (6) CAPAN-1, (7) D566, (8) U373, (9) C6 cells. HEK293 cells are positive and Jurkat are negative for FAP-1 expression. Cell lines tested express FAP-1 at varying levels. D566 (lane 7) were used for further study.

FIG. 6. FAP-1 and FAS co-immunoprecipitate. D566 cells were treated with FASL for indicated time and the lysates immunoprecipitated with either FAS antibody (top panel) or FAP-1 antibody (lower panel) and the association between the two proteins detected by blotting for the corresponding protein. FAP-1 associates with FAS in a FASL dependent manner. Constitutive association is seen in longer exposures and when FAP-1 is used to pull down FAS.

FIG. 7A-B. FAS is inducibly and reversibly tyrosine phosphorylated. (a) D566 cells were treated with FASL for 30 minutes and the lysates immunoprecipitated with FAS antibody and then immunoblotted for anti-phosphotyrosine (PY20 Ab). (b) FAP-1 immunoprecipitated from overexpressing cells dephosphorylates the biotinylated phospho-peptide (FASpY275) in an in vitro phosphatase assay. The level of dephosphorylation is relative to the vector condition as percent activity.

FIG. 8. Lysates immunoprecipitated with anti-FAS and anti-FAP-1 antibodies. These data provide a direct demonstration that FAS is tyrosine phosphorylated in response to FASL, and that endogenous FAP-1 dephosphorylates FAS.

### DETAILED DESCRIPTION OF THE EMBODIMENTS

The phosphatase PTPL1/FAP-1 is differentially expressed between brain tumor tissue and normal brain tissue, providing a specific marker for neoplastic cells, and a target for mediating the initiation and progression of brain tumors. Inhibition of PTPL1/FAP-1 gene and/or protein activity is advantageous in treating brain tumors, e.g. glioblastoma multiforme; ependymoma; glioma; astrocytoma; medulloblastoma; neuroglioma; oligodendroglioma; meningioma, etc., as well as other types of tumors. PTPL1/FAP-1 provides an excellent target for drug screening to identify pharmaceutically active agents, e.g. small organic molecules that interfere with tumor cell physiology and inhibit growth and replication of the tumor.

Screening methods may involve conducting various types of assays to identify agents that modulate the expression or activity of a PTPL1/FAP-1 gene or protein, or may involve screening for interfering with PTPL1/FAP-1 enzymatic activity in an in vitro, cell based, or in vivo system. Lead compounds identified during these screens can serve as the basis for the synthesis of more active analogs. Lead compounds and/or active analogs generated therefrom can be formulated into pharmaceutical compositions effective in treating brain tumors.

Therapeutic and prophylactic treatment methods for individuals suffering from, or at risk of a brain tumor, involve administering either a therapeutic or prophylactic amount of an agent that modulates the activity of a PTPL1/FAP-1 protein or gene, which may specifically bind to PTPL1/FAP-1 protein.

Without the invention being bound by the theory, the data provided herein suggest that over-expression of FAP-1 results in a resistance to apoptosis by the tumor cell. Defects in pathways that regulate apoptosis are involved in the growth of, for example, gliomas, and are in part responsible for resistance to adjuvant chemotherapy. Support of this comes from the observation that the tumor suppressor gene, p53, plays a role in both in the formation of low grade astrocytomas and in progression towards malignant glioma. p53 mutations occur in ⅓ of all high grades adult astrocytomas.

In glioblastoma, cytotoxic agents promote the expression of the death receptor, FAS, using both p53 dependent and independent pathways. FAS is a transmembrane receptor that plays a central role in programmed cell death. Activation of FAS induces cysteine proteases called caspases that cleave cellular proteins and ultimately cause cell death. Strategies to enhance death receptor pathways, by inducing FAS surface expression, have proven effective at overcoming resistance to apoptosis. However, despite cellular expression of FAS and FASL, gliomas are typically resistant to this form of cell death.

Protein tyrosine phosphatases (PTPs) are a promising class of signaling targets for disease intervention. Because most intracellular signaling involves reversible phosphorylation events, PTPs are central to the dynamic regulation of signaling cascades that underlie cell functions. FAS associated phosphatase (FAP-1, PTP-BAS, hPTP1 E, PTPL1) is a 270 kDa protein expressed in many tissues and cell lines. FAP-1 contains an ezrin-like cytoskeleton binding domain, an amino terminal leucine zipper motif, and six PSD95/Dlg/Z-1 homology (PDZ) domains. The PDZ domains of FAP-1 have been shown to bind to the cytosolic tail of Fas.

In studies using overexpression and RNA interference to modulate FAP-1, it has been found that FAP-1 inhibits trafficking of FAS to the cell surface. Elevated FAP-1 protein levels in some tumor cell lines and tissue correlates with resistance to Fas-induced apoptosis.

The examples provided herein demonstrate a role for FAP-1 in glioblastoma. FAP-1 mRNA and protein are specifically upregulated in glioblastoma tissue. By knocking-down FAP-1 expression using RNA interference technology, apoptosis of human glioblastoma cells is increased. This effect is blocked by the addition of neutralizing anti-FASL antibody to the cells. The data also demonstrate a functional interaction between FAP-1 and FAS, and demonstrate that FAP-1 directly dephosphorylates FAS as part of the downregulation of the apoptotic pathway.

### Disease Conditions

The present methods are applicable to brain tumors, particularly glioblastoma. In general, the goals of brain tumor treatments are to remove as many tumor cells as possible, e.g. with surgery, kill as many of the cells left behind after surgery as possible with radiation and/or chemotherapy, and put remaining tumor cells into a nondividing, quiescent state for as long as possible with radiation and chemotherapy. Careful imaging surveillance is a crucial part of medical care, because tumor regrowth requires alteration of current treatment, or, for patients in the observation phase, restarting treatment.

Brain tumors are classified according to the kind of cell from which the tumor seems to originate. Diffuse, fibrillary astrocytomas are the most common type of primary brain tumor in adults. These tumors are divided histopathologically into three grades of malignancy: World Health Organization (WHO) grade II astrocytoma, WHO grade III anaplastic astrocytoma and WHO grade IV glioblastoma multiforme (GBM). WHO grade II astocytomas are the most indolent of the diffuse astrocytoma spectrum. Astrocytomas display a remarkable tendency to infiltrate the surrounding brain, confounding therapeutic attempts at local control. These invasive abilities are often apparent in low-grade as well as high-grade tumors.

Glioblastoma multiforme is the most malignant stage of astrocytoma, with survival times of less than 2 years for most patients. Histologically, these tumors are characterized by dense cellularity, high proliferation indices, endothelial proliferation and focal necrosis. The highly proliferative nature of these lesions likely results from multiple mitogenic effects. One of the hallmarks of GBM is endothelial proliferation. A host of angiogenic growth factors and their receptors are found in GBMs.

There are subsets of astrocytomas, which may reflect the clinical heterogeneity observed in these tumors. These subsets include brain stem gliomas, which are a form of pediatric diffuse, fibrillary astrocytoma that often follow a malignant course. Brain stem GBMs share genetic features with those adult GBMs that affect younger patients. Pleomorphic xanthoastrocytoma (PXA) is a superficial, low-grade astrocytic tumor that predominantly affects young adults. While these tumors have a bizarre histological appearance, they are typically slow-growing tumors that may be amenable to surgical cure. Some PXAs, however, may recur as GBM. Pilocytic astrocytoma is the most common astrocytic tumor of childhood and differs clinically and histopathologically from the diffuse, fibrillary astrocytoma that affects adults. Pilocytic astrocytomas do not have the same genomic alterations as diffuse, fibrillary astrocytomas. Subependymal giant cell astrocytomas (SEGA) are periventricular, low-grade astrocytic tumors that are usually associated with tuberous sclerosis (TS), and are histologically identical to the so-called “candle-gutterings” that line the ventricles of TS patients. Similar to the other tumorous lesions in TS, these are slowly-growing and may be more akin to hamartomas than true neoplasms. Desmoplastic cerebral astrocytoma of infancy (DCAI) and desmoplastic infantile ganglioglioma (DIGG) are large, superficial, usually cystic, benign astrocytomas that affect children in the first year or two of life.

Oligodendrogliomas and oligoastrocytomas (mixed gliomas) are diffuse, usually cerebral tumors that are clinically and biologically most closely related to the diffuse, fibrillary astrocytomas. The tumors, however, are far less common than astrocytomas and have generally better prognoses than the diffuse astrocytomas. Oligodendrogliomas and oligoastrocytomas may progress, either to WHO grade III anaplastic oligodendroglioma or anaplastic oligoastrocytoma, or to WHO grade IV GBM. Thus, the genetic changes that lead to oligodendroglial tumors constitute yet another pathway to GBM.

Ependymomas are a clinically diverse group of gliomas that vary from aggressive intraventricular tumors of children to benign spinal cord tumors in adults. Transitions of ependymoma to GBM are rare. Choroid plexus tumors are also a varied group of tumors that preferentially occur in the ventricular system, ranging from aggressive supratentorial intraventricular tumors of children to benign cerebellopontine angle tumors of adults. Choroid plexus tumors have been reported occasionally in patients with Li-Fraumeni syndrome and von Hippel-Lindau (VHL) disease.

Medulloblastomas are highly malignant, primitive tumors that arise in the posterior fossa, primarily in children. Meningiomas are common intracranial tumors that arise in the meninges and compress the underlying brain. Meningiomas are usually benign, but some “atypical” meningiomas may recur locally, and some meningiomas are malignant and may invade the brain or metastasize. Atypical and malignant meningiomas are not as common as benign meningiomas. Schwannomas are benign tumors that arise on peripheral nerves. Schwannomas may arise on cranial nerves, particularly the vestibular portion of the eighth cranial nerve (vestibular schwannomas, acoustic neuromas) where they present as cerebellopontine angle masses. Hemangioblastomas are tumors of uncertain origin that are composed of endothelial cells, pericytes and so-called stromal cells. These benign tumors most frequently occur in the cerebellum and spinal cord of young adults. Multiple hemangioblastomas are characteristic of von Hippel-Lindau disease (VHL). Hemangiopericytomas (HPCs) are dural tumors, which may display locally aggressive behavior and may metastasize. The histogenesis of dural-based hemangiopericytoma (HPC) has long been debated, with some authors classifying it as a distinct entity and others classifying it as a subtype of meningioma.

The symptoms of both primary and metastatic brain tumors depend mainly on the location in the brain and the size of the tumor. Since each area of the brain is responsible for specific functions, the symptoms will vary a great deal. Tumors in the frontal lobe of the brain may cause weakness and paralysis, mood disturbances, difficulty in thinking, confusion and disorientation, and wide emotional mood swings. Parietal lobe tumors may cause seizures, numbness or paralysis, difficulty with handwriting, inability to perform simple mathematical problems, difficulty with certain movements, and loss of the sense of touch. Tumors in the occipital lobe can cause loss of vision in half of each visual field, visual hallucinations, and seizures. Temporal lobe tumors can cause seizures, perceptual and spatial disturbances, and receptive aphasia. If a tumor occurs in the cerebellum, the person may have ataxia, loss of coordination, headaches, and vomiting. Tumors in the hypothalamus may cause emotional changes, and changes in the perception of hot and cold. In addition, hypothalamic tumors may affect growth and nutrition in children. With the exception of the cerebellum, a tumor on one side of the brain causes symptoms and impairment on the opposite side of the body.

Other disorders of the nervous system that may be treated or imaged with the compositions of the present invention include, but are not limited to ischemic stroke, brain cancer, epilepsy, schizophrenia, depression, Alzheimer's Disease, Parkinson's Disease, Huntington's Chorea, traumatic head injury, dementia, coma, stupor, headache (and other neurological pain), vertigo, weakness, myasthenia gravis (and other disorders of the neuromuscular junction), ataxia and cerebellar disorders, cranial nerve disorders (such as Bell's Palsy), cerebrovascular disorders, infectious disorders including bacterial, fungal, viral and parasitic infections, multiple sclerosis, and other complications associated with pregnancy, medical illness, alcohol and substance abuse, toxins and metabolic deficiencies.

Compounds identified by the methods of the invention can be used therapeutically. As used herein, the term “treating” is used to refer to both prevention of disease, and treatment of pre-existing conditions. The prevention of symptoms is accomplished by administration of the compounds and pharmaceutical compositions of the invention prior to development of overt disease, e.g., to prevent the regrowth of tumors or prevent metastatic growth. Alternatively, the compounds and pharmaceutical compositions of the invention may be administered to a subject in need thereof to treat an ongoing disease, by stabilizing or improving the clinical symptoms of the patient.

The subject, or patient, may be from any mammalian species, e.g. primates, particularly humans; rodents, including mice, rats and hamsters; rabbits; equines; bovines; canines; felines; etc. Animal models are of interest for experimental investigations, providing a model for treatment of human disease.

Hyperproliferative disorders refers to excess cell proliferation, relative to that occurring with the same type of cell in the general population and/or the same type of cell obtained from a patient at an earlier time. The term denotes malignant as well as non-malignant cell populations. Such disorders have an excess cell proliferation of one or more subsets of cells, which often appear to differ from the surrounding tissue both morphologically and genotypically. The excess cell proliferation can be determined by reference to the general population and/or by reference to a particular patient, e.g. at an earlier point in the patient's life. Hyperproliferative cell disorders can occur in different types of animals and in humans, and produce different physical manifestations depending upon the affected cells.

Cancers of particular interest include carcinomas, e.g. colon, prostate, breast, melanoma, ductal, endometrial, stomach, dysplastic oral mucosa, invasive oral cancer, non-small cell lung carcinoma, transitional and squamous cell urinary carcinoma, etc.; neurological malignancies, e.g. neuroblastoma, gliomas, etc.; hematological malignancies, e.g. childhood acute leukaemia, non-Hodgkin's lymphomas, chronic lymphocytic leukaemia, malignant cutaneous T-cells, mycosis fungoides, non-MF cutaneous T-cell lymphoma, lymphomatoid papulosis, T-cell rich cutaneous lymphoid hyperplasia, bullous pemphigoid, discoid lupus erythematosus, lichen planus, etc.; sarcomas, melanomas, adenomas; benign lesions such as papillomas, and the like. Preferably excluded from carcinomas of interest are breast carcinomas, insulinomas, and glucagonomas.

### Identification of PTPL1/FAP-1 as a Tumor Target Gene

A genetic sequence that comprises all or a part of a cDNA sequence that is differentially expressed in brain tumor cells, particularly glioblastoma cells, relative to expression in normal, or non-disease conditions, is herein termed a “target gene”, which encodes a “target protein”. PTPL1/FAP-1 was identified as a target by creating subtracted and normalized cDNA libraries from glioblastoma tissues. The cDNAs from control and disease states were subjected to kinetic re-annealing hybridization during which normalization of transcript abundances and enrichment for low abundance transcripts occurs. Differential up- or down-regulated transcripts in tumors can be enriched by a subsequent “forward” or “reverse” subtraction step using a second driver cDNA as described in co-pending U.S. patent application Ser. No. 09/627,362, filed on Jul. 28, 2000.

Clones displaying a significant transcriptional induction and/or repression were sequenced and carried forward for expression profiling, using a variety of temporal, spatial and disease-related probe sets. Selected clones showing a significant transcriptional induction and/or repression were sequenced and functionally annotated in a proprietary database structure (See WO01/13105). Because large sequence fragments were utilized in the sequencing step, the data generated has a much higher fidelity and specificity than other approaches, such as SAGE. The resulting sequence information was compared to public databases using the BLAST (blastn) algorithm for DNA sequence comparisons and iterative-Smith Waterman analysis for protein sequence comparisons.

Transcripts that represent differentially expressed genes have been identified by utilizing a variety of methods, including differential screening, subtractive hybridization, differential display, or hybridization to an array comprising a plurality of gene sequences.

“Differential expression” as used herein refers to both quantitative as well as qualitative differences in the genes' temporal and/or tissue expression patterns. Thus, a differentially expressed gene may have its expression activated or inactivated in normal versus neuronal disease conditions, or in control versus experimental conditions. Such a qualitatively regulated gene will exhibit an expression pattern within a given tissue or cell type that is detectable in either control or tumor samples, but is not detectable in both. Detectable, as used herein, refers to an RNA expression pattern that is detectable via the standard techniques of differential display, reverse transcription-(RT-) PCR and/or Northern analyses, which are well known to those of skill in the art. Generally, differential expression means that there is at least a 20% change, and in other instances at least a 2-, 3-, 5- or 10-fold difference between disease and control tissue expression. The difference usually is one that is statistically significant, meaning that the probability of the difference occurring by chance (the P-value) is less than some predetermined level (e.g., 5%). Usually the confidence level (P value) is <0.05, more typically <0.01, and in other instances, <0.001.

Alternatively, a differentially expressed gene may have its expression modulated, i.e., quantitatively increased or decreased, in normal versus neuronal disease states, or under control versus experimental conditions. The difference in expression need only be large enough to be visualized via standard detection techniques as described above. Generally the difference in expression levels, measured by either the presence of mRNA or the protein product, will differ from basal levels (i.e. normal tissue) by at least about 2 fold, usually at least about 5 fold, and may be 10 fold, 100 fold, or more.

Once a sequence has been identified as differentially expressed, the sequence can be subjected to a functional validation process to determine whether the gene plays a role in tumor initiation, progression or maintenance. Such candidate genes can potentially be correlated with a wide variety of cellular states or activities. The term “functional validation” as used herein refers to a process whereby one determines whether modulation of expression or function of a candidate gene or set of such genes causes a detectable change in a cellular activity or cellular state for a reference cell, which can be a population of cells such as a tissue or an entire organism. The detectable change or alteration that is detected can be any activity carried out by the reference cell. Specific examples of activities or states in which alterations can be detected include, but are not limited to, phenotypic changes (e.g., cell morphology, cell proliferation, cell viability and cell death); cells acquiring resistance to a prior sensitivity or acquiring a sensitivity which previously did not exist; protein/protein interactions; cell movement; intracellular or intercellular signaling; cell/cell interactions; cell activation (e.g., T cell activation, B cell activation, mast cell degranulation); release of cellular components (e.g., hormones, chemokines and the like); and metabolic or catabolic reactions.

Identification of genes associated with PTPL1/FAP-1 in signaling pathways, which may be referred to as PTPL1/FAP-1 interactors, can be performed through physical association of gene products, or through database identification of known physiological pathways. Among the methods for detecting protein-protein association are co-immunoprecipitation, crosslinking and co-purification through gradients or chromatographic columns. The two-hybrid system detects the association of proteins in vivo, as described by Chien et al. (1991) Proc. Natl. Acad. Sci. USA 88:9578-9582. The two-hybrid system or related methodology may be used to screen activation domain libraries for proteins that interact with a known “bait” gene protein. Known interactors with PTPL1/FAP-1 include Fas receptor (Saras et al. (1997) J. Biol. Chem. 272:20979-20981); and PARG1 (Saras et al. (1997) J. Biol. Chem. 272:24333-24338).

A variety of options are available for functionally validating the physiological function of PTPL1/FAP-1 interactors in brain tumors. Such methods include in situ hybridization and immunocytochemistry to confirm expression of the interactor in relevant tissues; and methods such as interference RNA (RNAi) to confirm the role of an interactor in a cell. The functional role that a interactor plays in a cell can also be assessed using gene “knockout” approaches in which the gene encoding the interactor is deleted, modified, or inhibited on either a single or both alleles. The cells or animals can be optionally be reconstituted with a wild-type gene as part of a further analysis. Methods for the use of RNAi are described, for example, in co-pending patent application Ser. No. 10/027,807, herein incorporated by reference. A number of options are available to detect interference of interactor expression (i.e., to detect gene silencing). In general, inhibition in expression is detected by detecting a decrease in the level of the interactor protein, determining the level of mRNA transcribed from the gene and/or detecting a change in phenotype associated with gene expression. Additional functional validation can utilize calcium flux measurements, electrophysiology and pharmacological characterization.

As shown herein, PTPL1/FAP-1 is differentially expressed in glioblastoma. An exemplary PTPL1/FAP-1 molecule is the human polypeptide, the sequence of which may be obtained at Genbank, accession number X80289, and is published by Saras et al. (1994) J. Biol. Chem. 269 (39):24082-24089. For convenience, the sequence of the gene and protein are provided herein as SEQ ID NO:1 and SEQ ID NO:2, respectively.

PTPL1/FAP-1 has a wide tissue distribution, a 9.5-kilobase transcript being expressed in most tissues. Peptide antisera against PTPL1/FAP-1 specifically precipitate a protein with an apparent mass of 250 kDa. PTPL1/FAP-1 has a PTP domain located in the COOH terminus, and the protein has been shown to dephosphorylate substrates. In the non-enzymatic part of PTPL1/FAP-1, three different structural motifs can be identified. Two of these are often found in proteins at the interface between the plasma membrane and the cytoskeleton, i.e. a 300-amino acid domain with similarity to the band 4.1 superfamily, and a region consisting of five GLGF repeats, an 80-amino acid repeat found in a variety of cytoskeleton-associated proteins. In addition to these structures PTPL1/FAP-1 has a region that fulfills the criteria for a leucine zipper motif.

### Compound Screening

PTPL1/FAP-1 sequences are used in screening of candidate compounds, usually small organic molecules, for the ability to bind to and/or inhibit PTPL1/FAP-1 activity. Agents that inhibit PTPL1/FAP-1 are of interest as therapeutic agents for the treatment of brain tumors. Such compound screening may be performed using an in vitro model, a genetically altered cell or animal, or purified protein corresponding to PTPL1/FAP-1 or a fragment thereof. One can identify ligands or substrates that bind to, modulate or mimic the action of the encoded polypeptide.

In one embodiment of the invention, compounds useful in the treatment of tumors are assayed for an ability to inhibit the dephosphorylation of a substrate by FAP-1. The data provided herein demonstrates an association of FAS and FAP-1; and the direct dephosphorylation of FAS by FAP-1. Inhibitors of FAP-1 may prevent the specific dephosphorylation of the substrate; prevent the binding interaction between FAP-1 and FAS, and the like. Assays may comprise: FAP-1 or an active fragment thereof; a substrate of FAP-1; and a candidate agent for modulation of FAP-1 activity.

Substrates of interest include FAS protein, or a peptide derived therefrom. Fas antigen from human mouse cells is a protein containing a single transmembrane domain with a calculated molecular weight of 35,000. The sequence is publicly available, e.g. from Genbank, at accession no. M67454, or as described by Itoh et al. (1991) Cell 66(2):233-243. FAS shows structural homology with a number of cell-surface receptors, including tumor necrosis factor (TNF) receptors and low-affinity nerve growth factor receptor. When human Fas antigen is expressed in mouse cell lines, it can induce antibody-triggered apoptosis, or programmed cell death.

While the intact FAS protein may be used as a substrate, it is generally convenient to use a peptide derived therefrom. Such a peptide will comprise the site at which FAP-1 acts: the tyrosine corresponding to position 275 of the human protein. One of skill in the art will readily be able to substitute the sequence of, for example, monkey, mouse, rat, etc., sequence for the human peptide. A peptide of at least about 7, more usually at least about 8, preferably at least about about 9 amino acids in length, and including the sequence contiguous with Tyr 275 will be used. Exemplary of such peptides is the sequence KKEAYDTLI, where the tyrosine is phosphorylated. The peptide may be modified for ease of separation, detection, etc., e.g. by biotinylation, label with a detectable marker, fusion to a tag protein or peptide, and the like.

For example, a candidate agent may be contacted with an active fragment of FAP-1; a substrate; and a suitable buffer. Inhibitors of FAP-1 will prevent dephosphorylation of the substrate, which may be detected by any convenient method, e.g. binding to a phosphotyrosine specific antibody, etc.

Assays of interest include a time-resolved fluorescence resonance energy transfer assay. A candidate compound is added to a reaction mixture comprising buffer, FAP-1 enzyme and tagged substrate solution; and incubated for a period of time sufficient to allow for the enzyme to react. For detection, a labeled anti-phosphotyrosine antibody, a labeled binding agent specific for the substrate, and a stop buffer, e.g. orthovanadate is added to the reation. The labels are complementary pairs of a fluorescence energy transfer system, and the results are read by detecting fluorescence at the appropriate wavelengths.

In an alternative assay, an ELISA based immunoprecipitation is used, for example where an anti-phosphotyrosine antibody is used to capture substrate, and the level of precipitate is compared to the substrate in the absence or presence of a candidate agent. Alternatively, a binding agent specific for the substrate, e.g. an avidin/biotin system, may be used to precipitate or bind the substrate to a plate, and an antiphosphotyrosine antibody used for detection.

In other embodiments of the invention, compounds useful in the treatment of tumors are assayed by their effect on cells that express FAP-1, e.g. glioma cells. Such effects may include the ability of the cells to undergo programmed cell death; the activation of proteins involved in apoptosis, e.g. caspase 3, PARP, etc.; and the like. For example, a glioma cell expressing FAP-1 may be contacted with a candidate agent, in combination with a chemotherapeutic drug, or radiation, to induce apoptosis. The resistance of the glioma to induction of apoptosis provides a means of detecting activity of a candidate agent in modulating FAP-1 activity. Where cell death is not desired as an endpoint, methods known in the art may be used for quantitating mRNA or protein expression of proteins involved in the apoptosis pathway.

Screening may also detect the binding of FAP-1 and a substrate, e.g. FAS or a peptide derived therefrom. Such binding assays are readily performed using methods known in the art, and as decribed below.

Polypeptides useful in screening include PTPL1/FAP-1, FAS, and variants and derivatives thereof. Variant polypeptides can include amino acid (aa) substitutions, additions or deletions. The amino acid substitutions can be conservative amino acid substitutions or substitutions to eliminate non-essential amino acids, such as to alter a glycosylation site, a phosphorylation site or an acetylation site, or to minimize misfolding by substitution or deletion of one or more cysteine residues that are not necessary for function. Variants can be designed so as to retain or have enhanced biological activity of a particular region of the protein (e.g., a functional domain and/or, where the polypeptide is a member of a protein family, a region associated with a consensus sequence). Variants also include fragments of the polypeptides disclosed herein, particularly biologically active fragments and/or fragments corresponding to functional domains. Fragments of interest will typically be at least about 10 aa to at least about 15 aa in length, usually at least about 50 aa in length, and can be as long as 300 aa in length or longer, but will usually not exceed about 500 aa in length, where the fragment will have a contiguous stretch of amino acids that is identical to PTPL1/FAP-1, or a homolog or variant thereof.

Transgenic animals or cells derived therefrom are also used in compound screening. Transgenic animals may be made through homologous recombination, where the normal locus corresponding to a PTPL1/FAP-1 gene is altered. Alternatively, a nucleic acid construct is randomly integrated into the genome. Vectors for stable integration include plasmids, retroviruses and other animal viruses, YACs, and the like. A series of small deletions and/or substitutions may be made in the coding sequence to determine the role of different exons in enzymatic activity, oncogenesis, signal transduction, etc. Specific constructs of interest include antisense sequences that block expression of the targeted gene and expression of dominant negative mutations. A detectable marker, such as lac Z may be introduced into the locus of interest, where up-regulation of expression will result in an easily detected change in phenotype. One may also provide for expression of the target gene or variants thereof in cells or tissues where it is not normally expressed or at abnormal times of development, for example by overexpressing in neural cells. By providing expression of the target protein in cells in which it is not normally produced, one can induce changes in cell behavior.

Compound screening identifies agents that modulate function of PTPL1/FAP-1. Of particular interest are screening assays for agents that have a low toxicity for human cells. A wide variety of assays may be used for this purpose, including labeled in vitro protein-protein binding assays, in vitro phosphorylation assays, immunoassays for protein binding, and the like. Knowledge of the 3-dimensional structure of the encoded protein, derived from crystallization of purified recombinant protein, could lead to the rational design of small drugs that specifically inhibit activity. These drugs may be directed at specific domains.

The term “agent” as used herein describes any molecule, e.g. protein or pharmaceutical, with the capability of altering or mimicking the physiological function of PTPL1/FAP-1. Generally a plurality of assay mixtures are run in parallel with different agent concentrations to obtain a differential response to the various concentrations. Typically one of these concentrations serves as a negative control, i.e. at zero concentration or below the level of detection.

Candidate agents encompass numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs. Test agents can be obtained from libraries, such as natural product libraries or combinatorial libraries, for example.

Libraries of candidate compounds can also be prepared by rational design. (See generally, Cho et al., Pac. Symp. Biocompat. 305-16, 1998); Sun et al., J. Comput. Aided Mol. Des. 12:597-604, 1998); each incorporated herein by reference in their entirety). For example, libraries of phosphatase inhibitors can be prepared by syntheses of combinatorial chemical libraries (see generally DeWitt et al., Proc. Nat. Acad. Sci. USA 90:6909-13, 1993; International Patent Publication WO 94/08051; Baum, Chem. & Eng. News, 72:20-25,1994; Burbaum et al., Proc. Nat. Acad. Sci. USA 92:6027-31, 1995; Baldwin et al., J. Am. Chem. Soc. 117:5588-89, 1995; Nestler et al., J. Org. Chem. 59:4723-24, 1994; Borehardt et al., J. Am. Chem. Soc. 116:373-74, 1994; Ohlmeyer et al., Proc. Nat. Acad. Sci. USA 90:10922-26, all of which are incorporated by reference herein in their entirety.)

A “combinatorial library” is a collection of compounds in which the compounds comprising the collection are composed of one or more types of subunits. Methods of making combinatorial libraries are known in the art, and include the following: U.S. Pat. Nos. 5,958,792; 5,807,683; 6,004,617; 6,077,954; which are incorporated by reference herein. The subunits can be selected from natural or unnatural moieties. The compounds of the combinatorial library differ in one or more ways with respect to the number, order, type or types of modifications made to one or more of the subunits comprising the compounds. Alternatively, a combinatorial library may refer to a collection of “core molecules” which vary as to the number, type or position of R groups they contain and/or the identity of molecules composing the core molecule. The collection of compounds is generated in a systematic way. Any method of systematically generating a collection of compounds differing from each other in one or more of the ways set forth above is a combinatorial library.

A combinatorial library can be synthesized on a solid support from one or more solid phase-bound resin starting materials. The library can contain five (5) or more, preferably ten (10) or more, organic molecules that are different from each other. Each of the different molecules is present in a detectable amount. The actual amounts of each different molecule needed so that its presence can be determined can vary due to the actual procedures used and can change as the technologies for isolation, detection and analysis advance. When the molecules are present in substantially equal molar amounts, an amount of 100 picomoles or more can be detected. Preferred libraries comprise substantially equal molar amounts of each desired reaction product and do not include relatively large or small amounts of any given molecules so that the presence of such molecules dominates or is completely suppressed in any assay.

Combinatorial libraries are generally prepared by derivatizing a starting compound onto a solid-phase support (such as a bead). In general, the solid support has a commercially available resin attached, such as a Rink or Merrifield Resin. After attachment of the starting compound, substituents are attached to the starting compound. Substituents are added to the starting compound, and can be varied by providing a mixture of reactants comprising the substituents. Examples of suitable substituents include, but are not limited to, hydrocarbon substituents, e.g. aliphatic, alicyclic substituents, aromatic, aliphatic and alicyclic-substituted aromatic nuclei, and the like, as well as cyclic substituents; substituted hydrocarbon substituents, that is, those substituents containing nonhydrocarbon radicals which do not alter the predominantly hydrocarbon substituent (e.g., halo (especially chloro and fluoro), alkoxy, mercapto, alkylmercapto, nitro, nitroso, sulfoxy, and the like); and hetero substituents, that is, substituents which, while having predominantly hydrocarbyl character, contain other than carbon atoms. Suitable heteroatoms include, for example, sulfur, oxygen, nitrogen, and such substituents as pyridyl, furanyl, thiophenyl, imidazolyl, and the like. Heteroatoms, and typically no more than one, can be present for each carbon atom in the hydrocarbon-based substituents. Alternatively, there can be no such radicals or heteroatoms in the hydrocarbon-based substituent and, therefore, the substituent can be purely hydrocarbon.

Candidate agents of interest also include peptides and derivatives thereof, e.g. high affinity peptides or peptidomimetic substrates for PTPL1/FAP-1, particularly a substrate modified to act as an inhibitor. For example, tyrosine residues may be replaced with an inhibitory analog, see Liljebris et al. (2002) Bioorg Med Chem 10(10):3197-212; Liljebris et al. (2002) J Med Chem45(9):1785-98; and Jia et al. (2001) J Med Chem 44(26):4584-94. Such peptides may be resistant toward endo- and exo-proteolysis by gastric, pancreatic and small intestinal enzymes. Therefore selective oral inhibitors can be prepared by substituting tyrosine mimetics that act as mechanism based inhibitors of PTPL1/FAP-1 for reactive tyrosine or phosphotyrosine in a PTPL1/FAP-1 substrate.

Generally, peptide agents encompassed by the methods provided herein range in size from about 3 amino acids to about 100 amino acids, with peptides ranging from about 3 to about 25 being typical and with from about 3 to about 12 being more typical. Peptide agents can be synthesized by standard chemical methods known in the art (see, e.g., Hunkapiller et al., Nature 310:105-11, 1984; Stewart and Young, Solid Phase Peptide Synthesis, 2^{nd} Ed., Pierce Chemical Co., Rockford, Ill., (1984)), such as, for example, an automated peptide synthesizer. In addition, such peptides can be produced by translation from a vector having a nucleic acid sequence encoding the peptide using methods known in the art (see, e.g., Sambrook et al., Molecular Cloning, A Laboratory Manual, 3rd ed., Cold Spring Harbor Publish., Cold Spring Harbor, N.Y. (2001); Ausubel et al., Current Protocols in Molecular Biology, 4th ed., John Wiley and Sons, New York (1999); which are incorporated by reference herein).

Peptide libraries can be constructed from natural or synthetic amino acids. For example, a population of synthetic peptides representing all possible amino acid sequences of length N (where N is a positive integer), or a subset of all possible sequences, can comprise the peptide library. Such peptides can be synthesized by standard chemical methods known in the art (see, e.g., Hunkapiller et al., Nature 310:105-11, 1984; Stewart and Young, Solid Phase Peptide Synthesis, 2^{nd} Ed., Pierce Chemical Co., Rockford, Ill., (1984)), such as, for example, an automated peptide synthesizer. Nonclassical amino acids or chemical amino acid analogs can be used in substitution of or in addition into the classical amino acids. Non-classical amino acids include but are not limited to the D-isomers of the common amino acids, α-amino isobutyric acid, 4-aminobutyric acid, 2-amino butyric acid, γ-amino butyric acid, 6-amino hexanoic acid, 2-amino isobutyric acid, 3-amino propionic acid, ornithine, norleucine, norvaline, hydroxyproline, sarcosine, citrulline, cysteic acid, t-butylglycine, t-butylalanine, phenylglycine, cyclohexylalanine, β-alanine, selenocysteine, fluoro-amino acids, designer amino acids such as β-methy1 amino acids, C α-methyl amino acids, N α-methyl amino acids, and amino acid analogs in general. Furthermore, the amino acid can be D (dextrorotary) or L (levorotary).

Where the screening assay is a binding assay, one or more of the molecules may be joined to a label, where the label can directly or indirectly provide a detectable signal. Various labels include radioisotopes, fluorescers, chemiluminescers, enzymes, specific binding molecules, particles, e.g. magnetic particles, and the like. Specific binding molecules include pairs, such as biotin and streptavidin, digoxin and antidigoxin, etc. For the specific binding members, the complementary member would normally be labeled with a molecule that provides for detection, in accordance with known procedures.

A variety of other reagents may be included in the screening assay. These include reagents like salts, neutral proteins, e.g. albumin, detergents, etc that are used to facilitate optimal protein-protein binding and/or reduce non-specific or background interactions. Reagents that improve the efficiency of the assay, such as protease inhibitors, nuclease inhibitors, anti-microbial agents, etc. may be used. The components are added in any order that provides for the requisite binding. Incubations are performed at any suitable temperature, typically between 4 and 40° C. Incubation periods are selected for optimum activity, but may also be optimized to facilitate rapid high-throughput screening. Typically between 0.1 and 1 hours will be sufficient.

Preliminary screens can be conducted by screening for compounds capable of binding to PTPL1/FAP-1, as at least some of the compounds so identified are likely inhibitors. The binding assays usually involve contacting PTPL1/FAP-1 with one or more test compounds and allowing sufficient time for the protein and test compounds to form a binding complex. Any binding complexes formed can be detected using any of a number of established analytical techniques. Protein binding assays include, but are not limited to, methods that measure co-precipitation, co-migration on non-denaturing SDS-polyacrylamide gels, surface plasmon resonance and co-migration on Western blots (see, e.g., Bennet, J. P. and Yamamura, H. I. (1985) “Neurotransmitter, Hormone or Drug Receptor Binding Methods,” in Neurotransmitter Receptor Binding (Yamamura, H. I., et al., eds.), pp. 61-89.

Certain screening methods involve screening for a compound that modulates the expression of PTPL1/FAP-1. Such methods generally involve conducting cell-based assays in which test compounds are contacted with one or more cells expressing PTPL1/FAP-1 and then detecting and an increase in expression. Some assays are performed with tumor cells that express endogenous PTPL1/FAP-1 genes. Other expression assays are conducted with non-neuronal cells that express an exogenous PTPL1/FAP-1 gene.

The level of expression or activity can be compared to a baseline value. As indicated above, the baseline value can be a value for a control sample or a statistical value that is representative of expression levels for a control population. Expression levels can also be determined for cells that do not express PTPL1/FAP-1, as a negative control. Such cells generally are otherwise substantially genetically the same as the test cells. Various controls can be conducted to ensure that an observed activity is authentic including running parallel reactions with cells that lack the reporter construct or by not contacting a cell harboring the reporter construct with test compound. Compounds can also be further validated as described below.

Compounds that are initially identified by any of the foregoing screening methods can be further tested to validate the apparent activity. The basic format of such methods involves administering a lead compound identified during an initial screen to an animal that serves as a model for humans and then determining if PTPL1/FAP-1 is in fact upregulated. The animal models utilized in validation studies generally are mammals. Specific examples of suitable animals include, but are not limited to, primates, mice, and rats.

Active test agents identified by the screening methods described herein that inhibit PTPL1/FAP-1 activity and/or tumor growth can serve as lead compounds for the synthesis of analog compounds. Typically, the analog compounds are synthesized to have an electronic configuration and a molecular conformation similar to that of the lead compound. Identification of analog compounds can be performed through use of techniques such as self-consistent field (SCF) analysis, configuration interaction (Cl) analysis, and normal mode dynamics analysis. Computer programs for implementing these techniques are available. See, e.g., Rein et al., (1989) Computer-Assisted Modeling of Receptor-Ligand Interactions (Alan Liss, New York).

### Pharmaceutical Formulations

Compositions of agents that modulate expression and/or activity of PTPL1/FAP-1 find use in the treatment of gliomas and other tumors. The agents may be formulated for delivery to the brain. One strategy for drug delivery through the blood brain barrier (BBB) entails disruption of the BBB, either by osmotic means such as mannitol or leukotrienes, or biochemically by the use of vasoactive substances such as bradykinin. The potential for using BBB opening to target specific agents to brain tumors is also an option. A BBB disrupting agent can be co-administered with the therapeutic or imaging compositions of the invention when the compositions are administered by intravascular injection. Other strategies to go through the BBB may entail the use of endogenous transport systems, including carrier-mediated transporters such as glucose and amino acid carriers, receptor-mediated transcytosis for insulin or transferrin, and active efflux transporters such as p-glycoprotein. Active transport moieties may also be conjugated to the therapeutic or imaging compounds for use in the invention to facilitate transport across the epithelial wall of the blood vessel. Alternatively, drug delivery behind the BBB is by intrathecal delivery of therapeutics or imaging agents directly to the cranium, as through an Ommaya reservoir.

Pharmaceutical compositions can include, depending on the formulation desired, pharmaceutically-acceptable, non-toxic carriers of diluents, which are defined as vehicles commonly used to formulate pharmaceutical compositions for animal or human administration. The diluent is selected so as not to affect the biological activity of the combination. Examples of such diluents are distilled water, buffered water, physiological saline, PBS, Ringer's solution, dextrose solution, and Hank's solution. In addition, the pharmaceutical composition or formulation can include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents.

The composition can also include any of a variety of stabilizing agents, such as an antioxidant for example. When the pharmaceutical composition includes a polypeptide, the polypeptide can be complexed with various well-known compounds that enhance the in vivo stability of the polypeptide, or otherwise enhance its pharmacological properties (e.g., increase the half-life of the polypeptide, reduce its toxicity, enhance solubility or uptake). Examples of such modifications or complexing agents include sulfate, gluconate, citrate and phosphate. The polypeptides of a composition can also be complexed with molecules that enhance their in vivo attributes. Such molecules include, for example, carbohydrates, polyamines, amino acids, other peptides, ions (e.g., sodium, potassium, calcium, magnesium, manganese), and lipids.

Further guidance regarding formulations that are suitable for various types of administration can be found in Remington's Pharmaceutical Sciences, Mace Publishing Company, Philadelphia, Pa., 17th ed. (1985). For a brief review of methods for drug delivery, see, Langer, Science 249:1527-1533 (1990).

The pharmaceutical compositions can be administered for prophylactic and/or therapeutic treatments. Toxicity and therapeutic efficacy of the active ingredient can be determined according to standard pharmaceutical procedures in cell cultures and/or experimental animals, including, for example, determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices are preferred.

The data obtained from cell culture and/or animal studies can be used in formulating a range of dosages for humans. The dosage of the active ingredient typically lines within a range of circulating concentrations that include the ED₅₀ with low toxicity. The dosage can vary within this range depending upon the dosage form employed and the route of administration utilized.

The pharmaceutical compositions described herein can be administered in a variety of different ways. Examples include administering a composition containing a pharmaceutically acceptable carrier via oral, intranasal, rectal, topical, intraperitoneal, intravenous, intramuscular, subcutaneous, subdermal, transdermal, intrathecal, and intracranial methods.

For oral administration, the active ingredient can be administered in solid dosage forms, such as capsules, tablets, and powders, or in liquid dosage forms, such as elixirs, syrups, and suspensions. The active component(s) can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate. Examples of additional inactive ingredients that may be added to provide desirable color, taste, stability, buffering capacity, dispersion or other known desirable features are red iron oxide, silica gel, sodium lauryl sulfate, titanium dioxide, and edible white ink. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

The active ingredient, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen.

Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions.

The compositions of the invention may be administered using any medically appropriate procedure, e.g., intravascular (intravenous, intraarterial, intracapillary) administration, injection into the cerebrospinal fluid, intracavity or direct injection in the tumor. Intrathecal administration maybe carried out through the use of an Ommaya reservoir, in accordance with known techniques. (F. Balis et al., Am J. Pediatr. Hematol. Oncol. 11, 74, 76 (1989). For the imaging compositions of the invention, administration via intravascular injection is preferred for pre-operative visualization of the tumor. Post-operative visualization or visualization concurrent with an operation may be through intrathecal or intracavity administration, as through an Ommaya reservoir, or also by intravascular administration.

One method for administration of the therapeutic compositions of the invention is by deposition into the inner cavity of a cystic tumor by any suitable technique, such as by direct injection (aided by stereotaxic positioning of an injection syringe, if necessary) or by placing the tip of an Ommaya reservoir into a cavity, or cyst, for administration. Where the tumor is a solid tumor, the antibody may be administered by first creating a resection cavity in the location of the tumor. This procedure differs from an ordinary craniotomy and tumor resection only in a few minor respects. As tumor resection is a common treatment procedure, and is often indicated to relieve pressure, administration of the therapeutic compositions of the invention can be performed following tumor resection. Following gross total resection in a standard neurosurgical fashion, the cavity is preferable rinsed with saline until all bleeding is stopped by cauterization. Next the pia-arachnoid membrane, surrounding the tumor cavity at the surface, is cauterized to enhance the formation of fibroblastic reaction and scarring in the pia-arachnoid area. The result is the formation of an enclosed, fluid-filled cavity within the brain tissue at the location from where the tumor was removed. After the cyst has been formed, either the tip of an Ommaya reservoir or a micro catheter, which is connected to a pump device and allows the continues infusion of an antibody solution into the cavity, can be placed into the cavity. See, e.g., U.S. Pat. No. 5,558,852, incorporated fully herein by reference.

Alternatively, a convection-enhanced delivery catheter may be implanted directly into the tumor mass, into a natural or surgically created cyst, or into the normal brain mass. Such convection-enhanced pharmaceutical composition delivery devices greatly improve the diffusion of the composition throughout the brain mass. The implanted catheters of these delivery devices utilize high-flow microinfusion (with flow rates in the range of about 0.5 to 15.0 μl/minute), rather than diffusive flow, to deliver the therapeutic or imaging composition to the brain and/or tumor mass. Such devices are described in U.S. Pat. No. 5,720,720, incorporated fully herein by reference.

The effective amount of a therapeutic composition to be given to a particular patient will depend on a variety of factors, several of which will be different from patient to patient. A competent clinician will be able to determine an effective amount of a therapeutic agent to administer to a patient to retard the growth and promote the death of tumor cells, or an effective amount of an imaging composition to administer to a patient to facilitate the visualization of a tumor. Dosage of the antibody-conjugate will depend on the treatment of the tumor, route of administration, the nature of the therapeutics, sensitivity of the tumor to the therapeutics, etc. Utilizing LD₅₀ animal data, and other information available for the conjugated cytotoxic or imaging moiety, a clinician can determine the maximum safe dose for an individual, depending on the route of administration. For instance, an intravenously administered dose may be more than an intrathecally administered dose, given the greater body of fluid into which the therapeutic composition is being administered. Similarly, compositions which are rapidly cleared from the body may be administered at higher doses, or in repeated doses, in order to maintain a therapeutic concentration. Imaging moieties are typically less toxic than cytotoxic moieties and may be administered in higher doses in some embodiments. Utilizing ordinary skill, the competent clinician will be able to optimize the dosage of a particular therapeutic or imaging composition in the course of routine clinical trials.

The compositions can be administered to the subject in a series of more than one administration. For therapeutic compositions, regular periodic administration (e.g., every 2-3 days) will sometimes be required, or may be desirable to reduce toxicity. For therapeutic compositions that will be utilized in repeated-dose regimens, antibody moieties which do not provoke immune responses are preferred.

### Combination Therapies

Brain tumors tend to be heterogeneous in character, and pervasive throughout the brain tissue. This combination often makes them difficult to treat. In some cases, it may be preferred to use various combinations of therapeutic agents, in order to more fully target all of the cells exhibiting tumorigenic characteristics. Combinations of interest include administration of a PTPL1/FAP-1 inhibitor in conjunction with chemotherapeutic agents, and/or with chemosensitizers. Chemotherapeutic agents are known in the art, and include, for example, include alkylating agents, such as nitrogen mustards, e.g. mechlorethamine, cyclophosphamide, melphalan (L-sarcolysin), etc.; and nitrosoureas, e.g. carmustine (BCNU), lomustine (CCNU), semustine (methyl-CCNU), streptozocin, chlorozotocin, etc. Antimetabolite agents include pyrimidines, e.g. cytarabine (CYTOSAR-U), cytosine arabinoside, fluorouracil (5-FU), floxuridine (FUdR), etc.; purines, e.g. thioguanine (6-thioguanine), mercaptopurine (6-MP), pentostatin, fluorouracil (5-FU) etc.; and folic acid analogs, e.g. methotrexate, 10-propargyl-5,8-dideazafolate (PDDF, CB3717), 5,8-dideazatetrahydrofolic acid (DDATHF), leucovorin, etc. Other chemotherapeutic agents include azathioprine; brequinar; alkaloids and synthetic or semi-synthetic derivatives thereof, e.g. vincristine, vinblastine, vinorelbine, etc.; podophyllotoxins, e.g. etoposide, teniposide, etc.; antibiotics, e.g. anthracycline, daunorubicin hydrochloride (daunomycin, rubidomycin, cerubidine), idarubicin, doxorubicin, epirubicin and morpholino derivatives, etc.; phenoxizone biscyclopeptides, e.g. dactinomycin; basic glycopeptides, e.g. bleomycin; anthraquinone glycosides, e.g. plicamycin (mithrmycin); anthracenediones, e.g. mitoxantrone; azirinopyrrolo indolediones, e.g. mitomycin; and the like. Other chemotherapeutic agents include metal complexes, e.g. cisplatin (cis-DDP), carboplatin, etc.; ureas, e.g. hydroxyurea; and hydrazines, e.g. N-methylhydrazine.

Another combination of interest is the administration of a PTPL1/FAP-1 inhibitor in combination with radiation, and/or with radiation sensitizers. Radiosensitizers are compounds that, when combined with radiation, produce greater tumor cell kill than expected from a simple additive effect. Sensitizers include metronidazole, misonidazole, etanidazole, taxol, 5 fluorouracil, hydroxyurea, angiogenesis inhibitors, protein kinase C inhibitors, compounds such as motexafin gadolinium, and the like. Alternatively, the PTPL1/FAP-1 inhibitor may act as a sensitizing agent.

Radiation therapy for brain tumors is widely used, and will typically be used in combination with administration of a radiosensitizer. For example, ionizing radiation from X-rays or gamma rays may be delivered from an external source. Another technique for delivering radiation to cancer cells is internal radiotherapy, which places radioactive implants directly in the tumor so that the radiation dose is concentrated in a small area.

Such combination treatments may be by administering a PTPL1/FAP-1 modulating agent, and administering the blended therapeutic to the patient as described. The skilled administering physician will be able to take such factors as combined toxicity, and individual agent efficacy, into account when administering such combined agents. Additionally, those of skill in the art will be able to screen for potential cross-reaction with each other, in order to assure full efficacy of each agent.

Alternatively, several individual brain tumor protein target compositions may be administered simultaneously or in succession for a combined therapy. This may be desirable to avoid accumulated toxicity from several reagents, or to more closely monitor potential adverse reactions to the individual reagents. Thus, cycles such as where a therapeutic agent is administered on day one, followed by a second on day two, then a period with out administration, followed by re-administration of the therapeutics on different successive days, is comprehended within the present invention. Another combination therapy could include that of a small molecule drug and an antibody therapeutic against the individual brain tumor protein targets that are described in U.S. Pat. No. 6,455,026 and copending applicatons 09/983,000 10/329,258 and 10/328,544, incorporated fully herein by reference.

### Nucleic Acids

PTPL1/FAP-1 nucleic acids find use in diagnostic and therapeutic methods, for the recombinant production of the encoded polypeptide, and the like. The nucleic acids of the invention include nucleic acids having a high degree of sequence similarity or sequence identity to the sequence provided in SEQ ID NO:1. Sequence identity can be determined by hybridization under stringent conditions, for example, at 50° C. or higher and 0.1×SSC (9 mM NaCl/0.9 mM Na citrate). Hybridization methods and conditions are well known in the art, see, e.g., U.S. Pat. No. 5,707,829. Nucleic acids that are substantially identical to the provided nucleic acid sequence, e.g. allelic variants, genetically altered versions of the gene, splice variants etc., bind to the sequence provided in SEQ ID NO:1 under stringent hybridization conditions. Further specific guidance regarding the preparation of nucleic acids is provided by Fleury et al. (1997) Nature Genetics 15:269-272; Tartaglia et al., PCT Publication No. WO 96/05861; and Chen et al., PCT Publication No. WO 00/06087, each of which is incorporated herein in its entirety.

The gene corresponding to SEQ ID NO:1 may be obtained using various methods well known to those skilled in the art, including but not limited to the use of appropriate probes to detect the genes within an appropriate cDNA or genomic DNA library, antibody screening of expression libraries to detect cloned DNA fragments with shared structural features, direct chemical synthesis, and amplification protocols. Libraries are preferably prepared from cells or tissues of normal brains or brain tumors. Cloning methods are described in Berger and Kimmel, Guide to Molecular Cloning Techniques, Methods in Enzymology, 152, Academic Press, Inc. San Diego, Calif.; Sambrook, et al. (1989) Molecular Cloning—A Laboratory Manual (2nd ed) Vols. 1-3, Cold Spring Harbor Laboratory, Cold Spring Harbor Press, NY; and Current Protocols (1994), a joint venture between Greene Publishing Associates, Inc. and John Wiley and Sons, Inc.

The sequence obtained from clones containing partial coding sequences or non-coding sequences can be used to obtain the entire coding region by using the RACE method (Chenchik et al. (1995) CLONTECHniques 1: 5-8). Oligonucleotides can be designed from the partial clone's analyzed sequence and subsequently utilized to amplify a reverse transcribed mRNA encoding the entire coding sequence. Alternatively, probes can be used to screen cDNA libraries prepared from an appropriate cell or cell line in which the gene is transcribed. Once the target nucleic acid is identified, it can be isolated and cloned using well-known amplification techniques. Such techniques include, the polymerase chain reaction (PCR) the ligase chain reaction (LCR), Qβ-replicase amplification, the self-sustained sequence replication system (SSR) and the transcription based amplification system (TAS). Such methods include, those described, for example, in U.S. Pat. No. 4,683,202 to Mullis et al.; PCR Protocols A Guide to Methods and Applications (Innis et al. eds) Academic Press Inc. San Diego, Calif. (1990); Kwoh et al. (1989) Proc. Natl. Acad. Sci. USA 86: 1173; Guatelli et al. (1990) Proc. Natl. Acad. Sci. USA 87: 1874; Lomell et al. (1989) J. Clin. Chem. 35: 1826; Landegren et al. (1988) Science 241: 1077-1080; Van Brunt (1990) Biotechnology 8: 291-294; Wu and Wallace (1989) Gene 4: 560; and Barringer et al. (1990) Gene 89: 117.

As an alternative to cloning a nucleic acid, a suitable nucleic acid can be chemically synthesized. Direct chemical synthesis methods include, for example, the phosphotriester method of Narang et al. (1979) Meth. Enzymol. 68: 90-99; the phosphodiester method of Brown et al. (1979) Meth. Enzymol. 68: 109-151; the diethylphosphoramidite method of Beaucage et al. (1981) Tetra. Lett., 22: 1859-1862; and the solid support method of U.S. Pat. No. 4,458,066. Chemical synthesis produces a single stranded oligonucleotide. This can be converted into double stranded DNA by hybridization with a complementary sequence, or by polymerization with a DNA polymerase using the single strand as a template. While chemical synthesis of DNA is often limited to sequences of about 100 bases, longer sequences can be obtained by the ligation of shorter sequences. Alternatively, subsequences may be cloned and the appropriate subsequences cleaved using appropriate restriction enzymes.

The nucleic acids can be cDNAs or genomic DNAs, as well as fragments thereof. The term “cDNA” as used herein is intended to include all nucleic acids that share the arrangement of sequence elements found in native mature mRNA species, where sequence elements are exons and 3′ and 5′ non-coding regions. Normally mRNA species have contiguous exons, with the intervening introns, when present, being removed by nuclear RNA splicing, to create a continuous open reading frame encoding a polypeptide of the invention.

A genomic sequence of interest comprises the nucleic acid present between the initiation codon and the stop codon, as defined in the listed sequences, including all of the introns that are normally present in a native chromosome. It can further include the 3′ and 5′ untranslated regions found in the mature mRNA. It can further include specific transcriptional and translational regulatory sequences, such as promoters, enhancers, etc., including about 1 kb, but possibly more, of flanking genomic DNA at either the 5′ or 3′ end of the transcribed region. The genomic DNA flanking the coding region, either 3′ or 5′, or internal regulatory sequences as sometimes found in introns, contains sequences required for proper tissue, stage-specific, or disease-state specific expression, and are useful for investigating the up-regulation of expression in tumor cells.

Probes specific to the nucleic acid of the invention can be generated using the nucleic acid sequence disclosed in SEQ ID NO:1. The probes are preferably at least about 18 nt, 25 nt, 50 nt or more of the corresponding contiguous sequence of the sequence provided in SEQ ID NO:1, and are usually less than about 2, 1, or 0.5 kb in length. Preferably, probes are designed based on a contiguous sequence that remains unmasked following application of a masking program for masking low complexity. Double or single stranded fragments can be obtained from the DNA sequence by chemically synthesizing oligonucleotides in accordance with conventional methods, by restriction enzyme digestion, by PCR amplification, etc. The probes can be labeled, for example, with a radioactive, biotinylated, or fluorescent tag.

The nucleic acids of the subject invention are isolated and obtained in substantial purity, generally as other than an intact chromosome. Usually, the nucleic acids, either as DNA or RNA, will be obtained substantially free of other naturally-occurring nucleic acid sequences, generally being at least about 50%, usually at least about 90% pure and are typically “recombinant,” e.g., flanked by one or more nucleotides with which it is not normally associated on a naturally occurring chromosome.

The nucleic acids of the invention can be provided as a linear molecule or within a circular molecule, and can be provided within autonomously replicating molecules (vectors) or within molecules without replication sequences. Expression of the nucleic acids can be regulated by their own or by other regulatory sequences known in the art. The nucleic acids of the invention can be introduced into suitable host cells using a variety of techniques available in the art, such as transferrin polycation-mediated DNA transfer, transfection with naked or encapsulated nucleic acids, liposome-mediated DNA transfer, intracellular transportation of DNA-coated latex beads, protoplast fusion, viral infection, electroporation, gene gun, calcium phosphate-mediated transfection, and the like.

For use in amplification reactions, such as PCR, a pair of primers will be used. The exact composition of the primer sequences is not critical to the invention, but for most applications the primers will hybridize to the subject sequence under stringent conditions, as known in the art. It is preferable to choose a pair of primers that will generate an amplification product of at least about 50 nt, preferably at least about 100 nt. Algorithms for the selection of primer sequences are generally known, and are available in commercial software packages. Amplification primers hybridize to complementary strands of DNA, and will prime towards each other. For hybridization probes, it may be desirable to use nucleic acid analogs, in order to improve the stability and binding affinity. The term “nucleic acid” shall be understood to encompass such analogs.

### Polypeptides

PTPL1/FAP-1 polypeptides are of interest for screening methods, as reagents to raise antibodies, as therapeutics, and the like. Such polypeptides can be produced through isolation from natural sources, recombinant methods and chemical synthesis. In addition, functionally equivalent polypeptides may find use, where the equivalent polypeptide may contain deletions, additions or substitutions of amino acid residues that result in a silent change, thus producing a functionally equivalent differentially expressed on pathway gene product. Amino acid substitutions may be made on the basis of similarity in polarity, charge, solubility, hydrophobicity, hydrophilicity, and/or the amphipathic nature of the residues involved. “Functionally equivalent”, as used herein, refers to a protein capable of exhibiting a substantially similar in vivo activity as the polypeptide encoded by a PTPL1/FAP-1 polypeptide, as provided in SEQ ID NO: 2.

The polypeptides may be produced by recombinant DNA technology using techniques well known in the art. Methods that are well known to those skilled in the art can be used to construct expression vectors containing coding sequences and appropriate transcriptional/translational control signals. These methods include, for example, in vitro recombinant DNA techniques, synthetic techniques and in vivo recombination/genetic recombination. Alternatively, RNA capable of encoding the polypeptides of interest may be chemically synthesized.

Typically, the coding sequence is placed under the control of a promoter that is functional in the desired host cell to produce relatively large quantities of the gene product. An extremely wide variety of promoters are well-known, and can be used in the expression vectors of the invention, depending on the particular application. Ordinarily, the promoter selected depends upon the cell in which the promoter is to be active. Other expression control sequences such as ribosome binding sites, transcription termination sites and the like are also optionally included. Constructs that include one or more of these control sequences are termed “expression cassettes.” Expression can be achieved in prokaryotic and eukaryotic cells utilizing promoters and other regulatory agents appropriate for the particular host cell. Exemplary host cells include, but are not limited to, E. coli, other bacterial hosts, yeast, and various higher eukaryotic cells such as the COS, CHO and HeLa cells lines and myeloma cell lines.

In mammalian host cells, a number of viral-based expression systems may be used, including retrovirus, lentivirus, adenovirus, adeno-associated virus, and the like. In cases where an adenovirus is used as an expression vector, the coding sequence of interest can be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing differentially expressed or pathway gene protein in infected hosts.

Specific initiation signals may also be required for efficient translation of the genes. These signals include the ATG initiation codon and adjacent sequences. In cases where a complete gene, including its own initiation codon and adjacent sequences, is inserted into the appropriate expression vector, no additional translational control signals may be needed. However, in cases where only a portion of the gene coding sequence is inserted, exogenous translational control signals must be provided. These exogenous translational control signals and initiation codons can be of a variety of origins, both natural and synthetic. The efficiency of expression may be enhanced by the inclusion of appropriate transcription enhancer elements, transcription terminators, etc.

In addition, a host cell strain may be chosen that modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins. Appropriate cell lines or host systems can be chosen to ensure the correct modification and processing of the foreign protein expressed. To this end, eukaryotic host cells that possess the cellular machinery for proper processing of the primary transcript, glycosylation, and phosphorylation of the gene product may be used. Such mammalian host cells include but are not limited to CHO, VERO, BHK, HeLa, COS, MDCK, 293, 3T3, W138, etc.

For long-term, high-yield production of recombinant proteins, stable expression is preferred. For example, cell lines that stably express the differentially expressed or pathway gene protein may be engineered. Rather than using expression vectors that contain viral origins of replication, host cells can be transformed with DNA controlled by appropriate expression control elements, and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines that express the target protein. Such engineered cell lines may be particularly useful in screening and evaluation of compounds that affect the endogenous activity of the PTPL1/FAP-1 protein. A number of selection systems may be used, including but not limited to the herpes simplex virus thymidine kinase, hypoxanthine-guanine phosphoribosyltransferase, and adenine phosphoribosyltransferase genes. Antimetabolite resistance can be used as the basis of selection for dhfr, which confers resistance to methotrexate; gpt, which confers resistance to mycophenolic acid; neo, which confers resistance to the aminoglycoside G-418; and hygro, which confers resistance to hygromycin.

The polypeptide may be labeled, either directly or indirectly. Any of a variety of suitable labeling systems may be used, including but not limited to, radioisotopes such as ¹²⁵I; enzyme labeling systems that generate a detectable colorimetric signal or light when exposed to substrate; and fluorescent labels. Indirect labeling involves the use of a protein, such as a labeled antibody, that specifically binds to the polypeptide of interest. Such antibodies include but are not limited to polyclonal, monoclonal, chimeric, single chain, Fab fragments and fragments produced by a Fab expression library.

Once expressed, the recombinant polypeptides can be purified according to standard procedures of the art, including ammonium sulfate precipitation, affinity columns, ion exchange and/or size exclusivity chromatography, gel electrophoresis and the like (see, generally, R. Scopes, Protein Purification, Springer—Verlag, N.Y. (1982), Deutscher, Methods in Enzymology Vol. 182: Guide to Protein Purification., Academic Press, Inc. N.Y. (1990)).

As an option to recombinant methods, polypeptides and oligopeptides can be chemically synthesized. Such methods typically include solid-state approaches, but can also utilize solution based chemistries and combinations or combinations of solid-state and solution approaches. Examples of solid-state methodologies for synthesizing proteins are described by Merrifield (1964) J. Am. Chem. Soc. 85:2149; and Houghton (1985) Proc. Natl. Acad. Sci., 82:5132. Fragments of a PTPL1/FAP-1 protein can be synthesized and then joined together. Methods for conducting such reactions are described by Grant (1992) Synthetic Peptides: A User Guide, W. H. Freeman and Co., N.Y.; and in “Principles of Peptide Synthesis,” (Bodansky and Trost, ed.), Springer-Verlag, Inc. N.Y., (1993).

For various purposes, for example as an immunogen, the entire PTPL1/FAP-1 polypeptide or a fragment derived therefrom may be used. Preferably, one or more 8-30 amino acid peptide portions, e.g. of an extracellular domain may be utilized, with peptides in the range of 10-20 being a more economical choice. Custom-synthesized peptides in this range are available from a multitude of vendors, and can be order conjugated to KLH or BSA. Alternatively, peptides in excess of 30 amino acids may be synthesized by solid-phase methods, or may be recombinantly produced in a suitable recombinant protein production system. In order to ensure proper protein glycosylation and processing, an animal cell system (e.g., Sf9 or other insect cells, CHO or other mammalian cells) is preferred.

Similarly, in vivo models for human brain tumors, particularly nude mice/SCID mice model or rat models, have been described, for example see Antunes et al. (2000). J Histochem Cytochem 48, 847-58; Price et al. (1999) Clin Cancer Res 5, 845-54; and Senner et al. (2000). Acta Neuropathol (Berl) 99, 603-8. Once correct expression of the protein in the tumor model is verified, the effect of the candidate agent on the tumor masses in these models can be evaluated, wherein the ability of the agent to alter protein activity is indicated by a decrease in tumor growth or a reduction in the tumor mass. Thus, agents that exhibit the appropriate anti-tumor effect may be selected without direct knowledge of the particular biomolecular role of the protein in oncogenesis.

### Specific Binding Agents

Antibodies and other PTPL1/FAP-1 specific binding agents find use in, for example, diagnostic assays. The term “specific binding member” or “binding member” as used herein refers to a member of a specific binding pair, i.e. two molecules, usually two different molecules, where one of the molecules (i.e., first specific binding member) through chemical or physical means specifically binds to the other molecule (i.e., second specific binding member). Specific binding pairs of interest include carbohydrates and lectins; complementary nucleotide sequences; peptide ligands and receptor; effector and receptor molecules; hormones and hormone binding protein; enzyme cofactors and enzymes; enzyme inhibitors and enzymes; lipid and lipid-binding protein; etc. The specific binding pairs may include analogs, derivatives and fragments of the original specific binding member. For example, a receptor and ligand pair may include peptide fragments, chemically synthesized peptidomimetics, labeled protein, derivatized protein, etc.

In a preferred embodiment, the specific binding member is an antibody. The term “antibody” or “antibody moiety” is intended to include any polypeptide chain-containing molecular structure with a specific shape that fits to and recognizes an epitope, where one or more non-covalent binding interactions stabilize the complex between the molecular structure and the epitope. Antibodies of interest include “intrabodies”, as described, for example, in der Maur et al. (2002) J. Biol. Chem. 277:45075-45085. Intrabodies are single chain Fv fragments, and can be intracellularly expressed. Single chain antibodies (Fv, as described below) can be produced from phage libraries containing human variable regions. See U.S. Pat. No. 6,174,708.

Polyclonal antibodies can be raised by a standard protocol by injecting a production animal with an antigenic composition, formulated as described above. See, e.g., Harlow and Lane, Antibodies: A Laboratory Manual, Cold Spring Harbor Laboratory, 1988. In order to raise antibodies to particular epitopes, peptides derived from the full sequence may be utilized. Alternatively, in order to generate antibodies to relatively short peptide portions of the brain tumor protein target, a superior immune response may be elicited if the polypeptide is joined to a carrier protein, such as ovalbumin, BSA or KLH. Polyclonal antibodies specific for the polypeptide may be purified antisera by, for example, affinity chromatography using the polypeptide coupled to a suitable solid support.

Alternatively, for monoclonal antibodies, hybridomas may be formed by isolating the stimulated immune cells, such as those from the spleen of the inoculated animal. These cells are then fused to immortalized cells, such as myeloma cells or transformed cells, which are capable of replicating indefinitely in cell culture, thereby producing an immortal, immunoglobulin-secreting cell line. Many such cell lines are known to those skilled in the art. The resulting fused cells, or hybridomas, are cultured under conditions that allow for the survival of fused, but not unfused, cells and the resulting colonies screened for the production of the desired monoclonal antibodies. Colonies producing such antibodies are cloned, expanded, and grown so as to produce large quantities of antibody, see Kohler and Milstein, 1975 Nature 256:495 (the disclosures of which are hereby incorporated by reference).

In addition, the antibodies or antigen binding fragments may be produced by genetic engineering. In this technique, as with the standard hybridoma procedure, antibody-producing cells are sensitized to the desired antigen or immunogen. The messenger RNA isolated from the immune spleen cells or hybridomas is used as a template to make cDNA using PCR amplification. A library of vectors, each containing one heavy chain gene and one light chain gene retaining the initial antigen specificity, is produced by insertion of appropriate sections of the amplified immunoglobulin cDNA into the expression vectors. A combinatorial library is constructed by combining the heavy chain gene library with the light chain gene library. This results in a library of clones which co-express a heavy and light chain (resembling the Fab fragment or antigen binding fragment of an antibody molecule). The vectors that carry these genes are co-transfected into a host (e.g. bacteria, insect cells, mammalian cells, or other suitable protein production host cell.). When antibody gene synthesis is induced in the transfected host, the heavy and light chain proteins self-assemble to produce active antibodies that can be detected by screening with the antigen or immunogen.

Chimeric antibodies may be made by recombinant means by combining the murine variable light and heavy chain regions (VK and VH), obtained from a murine (or other animal-derived) hybridoma clone, with the human constant light and heavy chain regions, in order to produce an antibody with predominantly human domains. The production of such chimeric antibodies is well known in the art, and may be achieved by standard means (as described, e.g., in U.S. Pat. No. 5,624,659, incorporated fully herein by reference). Humanized antibodies are engineered to contain even more human-like immunoglobulin domains, and incorporate only the complementarity-determining regions of the animal-derived antibody. See, e.g., U.S. Pat. No. 6,187,287, incorporated fully herein by reference. Alternatively, polyclonal or monoclonal antibodies may be produced from animals that have been genetically altered to produce human immunoglobulins. Techniques for generating such animals, and deriving antibodies therefrom, are described in U.S. Pat. No. 6,162,963 and 6,150,584, incorporated fully herein by reference.

In addition to entire immunoglobulins (or their recombinant counterparts), immunoglobulin fragments comprising the epitope binding site (e.g., Fab′, F(ab′)₂, or other fragments) are useful as antibody moieties in the present invention. Fv fragments are heterodimers of the variable heavy chain domain (V_{H}) and the variable light chain domain (V_{L}). Recombinant Fvs in which V_{H} and V_{L} are connected by a peptide linker are typically stable, see, for example, Huston et al., Proc. Natl. Acad, Sci. USA 85:5879-5883 (1988) and Bird et al., Science 242:423-426 (1988), both fully incorporated herein, by reference.

Derivatized immunoglobulins with added chemical linkers, detectable moieties, such as fluorescent dyes, enzymes, substrates, chemiluminescent moieties and the like, or specific binding moieties, such as streptavidin, avidin, or biotin, and the like may be utilized in the methods and compositions of the present invention. For convenience, the term “antibody” or “antibody moiety” will be used throughout to generally refer to molecules which specifically bind to an epitope of PTPL1/FAP-1, although the term will encompass all immunoglobulins, derivatives, fragments, recombinant or engineered immunoglobulins, and modified immunoglobulins, as described above.

### Diagnostic and Prognostic Methods

The differential expression of PTPL1/FAP-1 gene and/or gene product in tumors indicates that it can serve as a marker for diagnosis, for imaging, as well as for therapeutic applications. In general, such diagnostic methods involve detecting an elevated level of expression of the PTPL1/FAP-1 gene transcript or gene product in the cells or tissue of an individual or a sample therefrom. A variety of different assays can be utilized to detect an increase in gene expression, including both methods that detect gene transcript and protein levels. More specifically, the diagnostic and prognostic methods disclosed herein involve obtaining a sample from an individual and determining at least qualitatively, and preferably quantitatively, the level of PTPL1/FAP-1 gene product expression in the sample. Usually this determined value or test value is compared against some type of reference or baseline value.

In one embodiment of the invention, a reagent for diagnosis is a substrate for PTPL1/FAP-1 enzymatic activity, where the substrate undergoes a detectable change in the presence of active PTPL1/FAP-1. Of interest are substrate specific biomarkers, which are imaging agents that are activated upon the action of the enzyme in the cell, and provide a functional measure of activity. Agents of interest usually include two moieties, a substrate moiety and a detection moiety. The substrate moiety is a group that is a substrate, preferably a specific substrate, for PTPL1/FAP-1. The substrate moiety may include a phosphate group or analog thereof for PTPL1/FAP-1 phosphatase activity. The detection moiety includes any group that is activated directly or indirectly by the enzymatic modification of the substrate. Other diagnostic agents of interest include nucleic acids complementary to PTPL1/FAP-1 sequences, or binding members, such as antibodies, that are specific for PTPL1/FAP-1 polypeptides.

Diagnostic agents are used to screen patient samples for increased expression of the PTPL1/FAP-1 mRNA or protein, or for the presence of amplified DNA in the cell. Samples can be obtained from a variety of sources. Samples are typically obtained from a human subject. However, the methods can also be utilized with samples obtained from various other mammals, such as primates, e.g. apes and chimpanzees, mice, cats, rats, and other animals. Such samples are referred to as a patient sample.

Samples can be obtained from the tissues or fluids of an individual, as well as from cell cultures or tissue homogenates. For example, samples can be obtained from spinal fluid, or tumor biopsy samples. Also included in the term are derivatives and fractions of such cells and fluids. Samples can also be derived from in vitro cell cultures, including the growth medium, recombinant cells and cell components. Diagnostic samples are collected from an individual that has, or is suspected to have, a brain tumor. The presence of specific markers is useful in identifying and staging the tumor.

Nucleic Acid Screening Methods

Some of the diagnostic and prognostic methods that involve the detection of PTPL1/FAP-1 gene transcripts begin with the lysis of cells and subsequent purification of nucleic acids from other cellular material, particularly mRNA transcripts. A nucleic acid derived from an mRNA transcript refers to a nucleic acid for whose synthesis the mRNA transcript, or a subsequence thereof, has ultimately served as a template. Thus, a cDNA reverse transcribed from an mRNA, an RNA transcribed from that cDNA, a DNA amplified from the cDNA, an RNA transcribed from the amplified DNA, are all derived from the mRNA transcript and detection of such derived products is indicative of the presence and/or abundance of the original transcript in a sample.

A number of methods are available for analyzing nucleic acids for the presence of a specific sequence, e.g. upregulated or downregulated expression. The nucleic acid may be amplified by conventional techniques, such as the polymerase chain reaction (PCR), to provide sufficient amounts for analysis. The use of the polymerase chain reaction is described in Saiki et al. (1985) Science 239:487, and a review of techniques may be found in Sambrook, et al. Molecular Cloning: A Laboratory Manual, CSH Press 1989, pp.14.2-14.33.

A detectable label may be included in an amplification reaction. Suitable labels include fluorochromes, e.g. ALEXA dyes (available from Molecular Probes, Inc.); fluorescein isothiocyanate (FITC), rhodamine, Texas Red, phycoerythrin, allophycocyanin,6-carboxyfluorescein(6-FAM),2,7-dimethoxy-4,5-dichloro-6-carboxyfluorescein (JOE), 6-carboxy-X-rhodamine (ROX), 6-carboxy-2,4,7,4,7-hexachlorofluorescein (HEX), 5-carboxyfluorescein (5-FAM) or N,N,N,N-tetramethyl-6-carboxyrhodamine (TAMRA), radioactive labels, e.g. ³²P, ³⁵S, ³H; etc. The label may be a two stage system, where the amplified DNA is conjugated to biotin, haptens, etc. having a high affinity binding partner, e.g. avidin, specific antibodies, etc., where the binding partner is conjugated to a detectable label. The label may be conjugated to one or both of the primers. Alternatively, the pool of nucleotides used in the amplification is labeled, so as to incorporate the label into the amplification product.

The sample nucleic acid, e.g. amplified, labeled, cloned fragment, etc. is analyzed by one of a number of methods known in the art. Probes may be hybridized to northern or dot blots, or liquid hybridization reactions performed. The nucleic acid may be sequenced by dideoxy or other methods, and the sequence of bases compared to a wild-type sequence. Single strand conformational polymorphism (SSCP) analysis, denaturing gradient gel electrophoresis(DGGE), and heteroduplex analysis in gel matrices are used to detect conformational changes created by DNA sequence variation as alterations in electrophoretic mobility. Fractionation is performed by gel or capillary electrophoresis, particularly acrylamide or agarose gels.

In situ hybridization methods are hybridization methods in which the cells are not lysed prior to hybridization. Because the method is performed in situ, it has the advantage that it is not necessary to prepare RNA from the cells. The method usually involves initially fixing test cells to a support (e.g., the flat surface of a microscope slide or the walls of a microtiter well) and then permeabilizing the cells with an appropriate permeabilizing solution. A solution containing labeled probes is then contacted with the cells and the probes allowed to hybridize. Excess probe is digested, washed away and the amount of hybridized probe measured. This approach is described in greater detail by Nucleic Acid Hybridization: A Practical Approach (Hames, et al., eds., 1987).

A variety of so-called “real time amplification” methods or “real time quantitative PCR” methods can also be utilized to determine the quantity of mRNA present in a sample. Such methods involve measuring the amount of amplification product formed during an amplification process. Fluorogenic nuclease assays are one specific example of a real time quantitation method that can be used to detect and quantitate transcripts. In general such assays continuously measure PCR product accumulation using a dual-labeled fluorogenic oligonucleotide probe—an approach frequently referred to in the literature simply as the “TaqMan” method. Additional details regarding the theory and operation of fluorogenic methods for making real time determinations of the concentration of amplification products are described, for example, in U.S. Pat. No. 5,210,015 to Gelfand, U.S. Pat. No. 5,538,848 to Livak, et al., and U.S. Pat. No. 5,863,736 to Haaland, each of which is incorporated by reference in its entirety.

Polypeptide Screening Methods

Screening for expression of the subject sequences may be based on the functional or antigenic characteristics of the protein. Functional assays include the detection of PTPL1/FAP-1 enzymatic activity through the use of a substrate specific biomarker. Various immunoassays designed to detect polymorphisms in PTPL1/FAP-1 may be used in screening. Detection may utilize staining of cells or histological sections, performed in accordance with conventional methods, using antibodies or other specific binding members that specifically bind to PTPL1/FAP-1 polypeptide. The antibodies or other specific binding members of interest are added to a cell sample, and incubated for a period of time sufficient to allow binding to the epitope, usually at least about 10 minutes. The antibody may be labeled with radioisotopes, enzymes, fluorescers, chemiluminescers, or other labels for direct detection. Alternatively, a second stage antibody or reagent is used to amplify the signal. Such reagents are well known in the art. For example, the primary antibody may be conjugated to biotin, with horseradish peroxidase-conjugated avidin added as a second stage reagent. Final detection uses a substrate that undergoes a color change in the presence of the peroxidase. The absence or presence of antibody binding may be determined by various methods, including flow cytometry of dissociated cells, microscopy, radiography, scintillation counting, etc.

An alternative method for diagnosis depends on the in vitro detection of binding between antibodies and the polypeptide corresponding to a sequence of SEQ ID NO:2 in a lysate. Measuring the concentration of the target protein in a sample or fraction thereof may be accomplished by a variety of specific assays. A conventional sandwich type assay may be used. For example, a sandwich assay may first attach specific antibodies to an insoluble surface or support. The particular manner of binding is not crucial so long as it is compatible with the reagents and overall methods of the invention. They may be bound to the plates covalently or non-covalently, preferably non-covalently.

The insoluble supports may be any compositions to which polypeptides can be bound, which is readily separated from soluble material, and which is otherwise compatible with the overall method. The surface of such supports may be solid or porous and of any convenient shape. Examples of suitable insoluble supports to which the receptor is bound include beads, e.g. magnetic beads, membranes and microtiter plates. These are typically made of glass, plastic (e.g. polystyrene), polysaccharides, nylon or nitrocellulose. Microtiter plates are especially convenient because a large number of assays can be carried out simultaneously, using small amounts of reagents and samples.

Patient sample lysates are then added to separately assayable supports (for example, separate wells of a microtiter plate) containing antibodies. Preferably, a series of standards, containing known concentrations of the test protein is assayed in parallel with the samples or aliquots thereof to serve as controls. Preferably, each sample and standard will be added to multiple wells so that mean values can be obtained for each. The incubation time should be sufficient for binding. After incubation, the insoluble support is generally washed of non-bound components. After washing, a solution containing a second antibody is applied. The antibody will bind to one of the proteins of interest with sufficient specificity such that it can be distinguished from other components present. The second antibodies may be labeled to facilitate direct, or indirect quantification of binding. In a preferred embodiment, the antibodies are labeled with a covalently bound enzyme capable of providing a detectable product signal after addition of suitable substrate. Examples of suitable enzymes for use in conjugates include horseradish peroxidase, alkaline phosphatase, malate dehydrogenase and the like. Where not commercially available, such antibody-enzyme conjugates are readily produced by techniques known to those skilled in the art. The incubation time should be sufficient for the labeled ligand to bind available molecules.

After the second binding step, the insoluble support is again washed free of non-specifically bound material, leaving the specific complex formed between the target protein and the specific binding member. The signal produced by the bound conjugate is detected by conventional means. Where an enzyme conjugate is used, an appropriate enzyme substrate is provided so a detectable product is formed.

Other immunoassays are known in the art and may find use as diagnostics. Ouchterlony plates provide a simple determination of antibody binding. Western blots may be performed on protein gels or protein spots on filters, using a detection system specific for the targeted polypeptide, conveniently using a labeling method as described for the sandwich assay.

In some cases, a competitive assay will be used. In addition to the patient sample, a competitor to the targeted protein is added to the reaction mix. The competitor and the target compete for binding to the specific binding partner. Usually, the competitor molecule will be labeled and detected as previously described, where the amount of competitor binding will be proportional to the amount of target protein present. The concentration of competitor molecule will be from about 10 times the maximum anticipated protein concentration to about equal concentration in order to make the most sensitive and linear range of detection.

### Therapeutic/Prophylactic Treatment Methods

Agents that modulate activity of a PTPL1/FAP-1 gene or protein provide a point of therapeutic or prophylactic intervention, particularly agents that inhibit or upregulate activity of the polypeptide, or expression of the gene. Numerous agents are useful in modulating this activity, including agents that directly modulate expression, e.g. expression vectors, small interfering RNA, antisense specific for the targeted polypeptide; and agents that act on the protein, e.g. specific antibodies and analogs thereof, small organic molecules that block catalytic activity, etc.

Methods can be designed to selectively deliver nucleic acids to certain cells. Examples of such cells include, neurons, microglia, astrocytes, endothelial cells, oligodendrocytes, etc. Certain treatment methods are designed to selectively express an expression vector to neurons and/or target the nucleic acid for delivery to nerve cells (microglia, astrocytes, endothelial cells, oligodendrocytes). One technique for achieving selective expression in nerve cells is to operably link the coding sequence to a promoter that is primarily active in nerve cells. Examples of such promoters include, but are not limited to, prion protein promoter, calcium-calmodulin dependent protein kinase promoter. Alternatively, or in addition, the nucleic acid can be administered with an agent that targets the nucleic acid to nerve cells. For instance, the nucleic acid can be administered with an antibody that specifically binds to a cell-surface antigen on the nerve cells or a ligand for a receptor on neuronal cells.

When liposomes are utilized, substrates that bind to a cell-surface membrane protein associated with endocytosis can be attached to the liposome to target the liposome to nerve cells and to facilitate uptake. Examples of proteins that can be attached include capsid proteins or fragments thereof that bind to nerve cells, antibodies that specifically bind to cell-surface proteins on nerve cells that undergo internalization in cycling and proteins that target intracellular localizations within nerve cells (see, e.g., Wu et al. (1987) J. Biol. Chem. 262:4429-4432; and Wagner, et al. (1990) Proc. Natl. Acad. Sci. USA 87:3410-3414). Gene marking and gene therapy protocols are reviewed by Anderson et al. (1992) Science 256:808-813. Various other delivery options can also be utilized. For instance, a nucleic acid containing a sequence of interest can be injected directly into the cerebrospinal fluid. Alternatively, such nucleic acids can be administered by intraventricular injections.

Antisense or siRNA molecules can be used to down-regulate expression in cells. The antisense reagent may be antisense oligonucleotides (ODN), particularly synthetic ODN having chemical modifications from native nucleic acids, or nucleic acid constructs that express such antisense molecules as RNA. The antisense sequence is complementary to the mRNA of the targeted gene, and inhibits expression of the targeted gene products. Antisense molecules inhibit gene expression through various mechanisms, e.g. by reducing the amount of mRNA available for translation, through activation of RNAse H, or steric hindrance. One or a combination of antisense molecules may be administered, where a combination may comprise multiple different sequences.

Antisense molecules may be produced by expression of all or a part of the target gene sequence in an appropriate vector, where the transcriptional initiation is oriented such that an antisense strand is produced as an RNA molecule. Alternatively, the antisense molecule is a synthetic oligonucleotide. Antisense oligonucleotides will generally be at least about 7, usually at least about 12, more usually at least about 20 nucleotides in length, and not more than about 500, usually not more than about 50, more usually not more than about 35 nucleotides in length, where the length is governed by efficiency of inhibition, specificity, including absence of cross-reactivity, and the like. It has been found that short oligonucleotides, of from 7 to 8 bases in length, can be strong and selective inhibitors of gene expression (see Wagner et al. (1996) Nature Biotechnology 14:840-844).

A specific region or regions of the endogenous sense strand mRNA sequence is chosen to be complemented by the antisense sequence. Selection of a specific sequence for the oligonucleotide may use an empirical method, where several candidate sequences are assayed for inhibition of expression of the target gene in vitro or in an animal model. A combination of sequences may also be used, where several regions of the mRNA sequence are selected for antisense complementation.

Antisense oligonucleotides may be chemically synthesized by methods known in the art (see Wagner et al. (1993) supra. and Milligan et al., supra.) Preferred oligonucleotides are chemically modified from the native phosphodiester structure, in order to increase their intracellular stability and binding affinity. A number of such modifications have been described in the literature, which alter the chemistry of the backbone, sugars or heterocyclic bases.

Among useful changes in the backbone chemistry are phosphorothioates; phosphorodithioates, where both of the non-bridging oxygens are substituted with sulfur; phosphoroamidites; alkyl phosphotriesters and boranophosphates. Achiral phosphate derivatives include 3′-O′-5′-S-phosphorothioate, 3′-S-5′-O-phosphorothioate, 3′-CH2-5′-O-phosphonate and 3′-NH-5′-O-phosphoroamidate. Peptide nucleic acids replace the entire ribose phosphodiester backbone with a peptide linkage. Sugar modifications are also used to enhance stability and affinity. The alpha.-anomer of deoxyribose may be used, where the base is inverted with respect to the natural .beta.-anomer. The 2′-OH of the ribose sugar may be altered to form 2′-O-methyl or 2′-O-allyl sugars, which provides resistance to degradation without comprising affinity. Modification of the heterocyclic bases must maintain proper base pairing. Some useful substitutions include deoxyuridine for deoxythymidine; 5-methyl-2′-deoxycytidine and 5-bromo-2′-deoxycytidine for deoxycytidine. 5-propynyl-2′-deoxyuridine and 5-propynyl-2′-deoxycytidine have been shown to increase affinity and biological activity when substituted for deoxythymidine and deoxycytidine, respectively.

Experimental

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

### EXAMPLE 1

### Identification of Differentially Expressed Sequences

Brain Tumors: Tumor tissue, confirmed as glioblastoma grade IV by neuropathology, from an unknown patient was snap frozen in the operation hall and served as experimental sample. Human whole brain tissue (Clontech Laboratories, Palo Alto, USA) served as control sample. Poly-A⁺ RNA prepared from the cells was converted into double-stranded cDNA (dscDNA) and normalized as described in co-pending U.S. patent application Ser. No. 09/627,362, filed on Jul. 28, 2000. Subtractive hybridization was carried out using the dscDNA from tumors with an excess of dscDNA prepared from the same region of a non-cancerous brain. Differentially expressed gene fragments were cloned into a plasmid vector, and the resulting library was transformed into E. coli cells. Inserts of recombinant clones were amplified by the polymerase chain reaction (PCR). The PCR products (fragments of 200-2000 bp in size) were sequenced using an oligonucleotide complementary to common vector sequences. The resulting sequence information was compared to public databases using the BLAST (blastn) and Smith Waterman algorithm. The differentially expressed sequence thus identified is listed in SEQ ID NO: 1.

Quantitative Real Time PCR. Total RNA from normal brain tissue samples and GBM tumor samples was isolated with Trizol (Gibco BRL) according to the manufacturer's instructions. SYBR Green real-time PCR amplifications were performed in an iCycler Real-Time Detection System (Bio-Rad Laboratories, Hercules, Calif.). The reactions were carried out in a 96-well plate in a 25-μl reaction volume containing 12.5 μl of 2× SYBR Green Master Mix (PE Applied Biosystems), a 0.9 μM concentration of each forward and reverse primer, 200 ng of total cDNA and supplemented to 25 μl with nuclease-free H₂O (Promega). Primers were designed using Primer3 developed by the Whitehead Institute for Biomedical Research and the primers (Operon Technologies, Alameda, Calif.) concentrations were optimized for use with the SYBR green PCR master mix reagents kit. The sizes of the amplicons were checked by running out the PCR product on a 1.5% agarose gel. The thermal profile for all SYBR Green PCRs was 50° C. for 2 minutes and 95° C. for 10 minutes, followed by 45 cycles of 95° C. for 15 seconds, 60° C. for 30 seconds followed by 72° C. for 40 seconds. The critical threshold cycle (Ct) is defined as the cycle at which the fluorescence becomes detectable above background and is inversely proportional to the logarithm of the initial number of template molecules. A standard curve was plotted for each primers set with Ct values obtained from amplification of 10-fold dilutions of cDNA obtained from whole brain. The standard curves are used to calculate the PCR efficiency of the primer set.

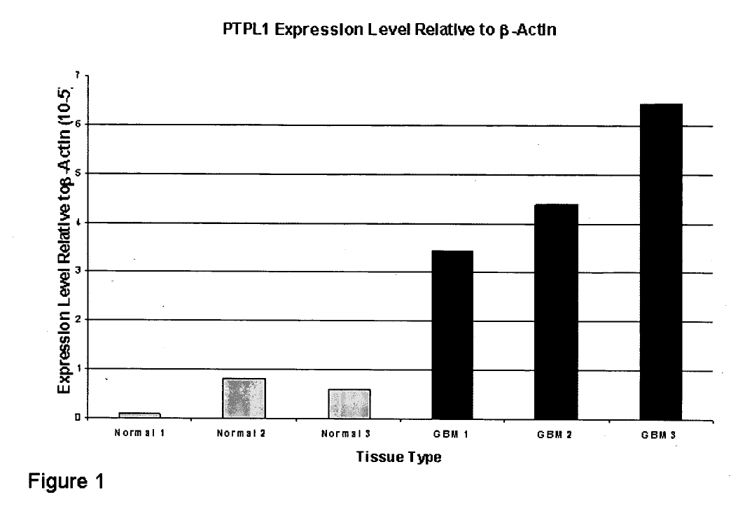

As an endogenous reference, glyceraldehydes-3-phosphate dehydrogenase (GAPDH) was used, recently demonstrated to be a suitable control gene for studying brain injury with real-time RT-PCR (Harrison et al, 2000). All PCR reactions performed in duplicate. Quantification was performed using the comparative cycle threshold (CT) method, where CT is defined as the cycle number at which fluorescence reaches a set threshold value. The target transcript was normalized to an endogenous reference (simultaneous triplicate GAPDH reactions), and relative differences were calculated using the PCR efficiencies according to Pfaffl (Nucleic Acids Research, 2001). In order to demonstrate that PTPL1/FAP-1 is upregulated in GBM versus normal brain tissue we obtained surgically removed GBM tumor samples and normal control brain tissue from various sources. Total RNA was extracted from these samples using established methods and cDNA was generated for use in the real time quantitative PCR procedure. These studies demonstrate that PTPL1/FAP-1 mRNA is upregulated in GBM tumor tissue (FIG. 1). PTPL1/FAP-1 mRNA is relatively low in abundance in normal brain tissue and therefore PTPL1/FAP-1 expression is a good diagnostic marker of GBM and a specific target for GBM therapies.

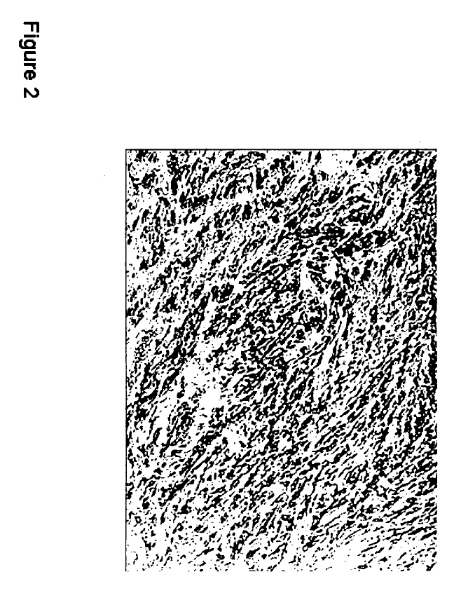

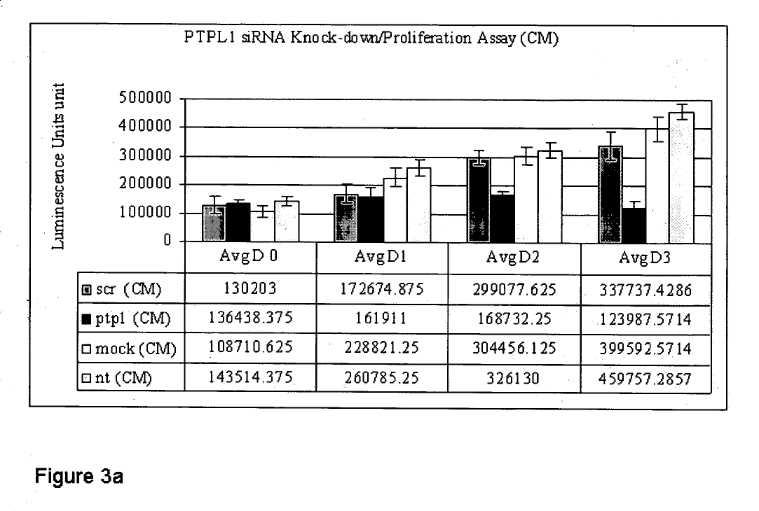

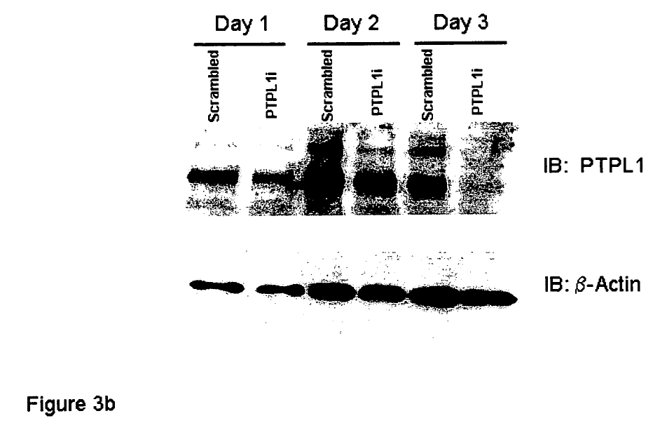

Immunohistochemistry. For immunohistochemistry, human normal brain and tumor sections were used. The human cancer tissue array slides were used to evaluate the tissue specific expression with antibodies to PTPL1/FAP-1. These paraffin embeded tissue array slides were dewaxed, washed in water and treated with target retrieval procedures. Conventional immunhistochemical reactions were then carried out using an anti-PTPL1/FAP-1 antibody. Tissue sections were analyzed using light microscopy for localization of staining, as well as intensity and tissue section ultrastructure. The protein expression level and localization of PTPL1/FAP-1 in tumor tissue was determined. These studies demonstrate that PTPL1/FAP-1 protein is expressed in glioblastoma tissue and other tumor tissues (FIG. 2). The expression of PTPL1/FAP-1 in GBM indicates that this protein is a good diagnostic marker of tumors, including GBM, and a specific target for anti-tumor therapies.

Cell Proliferation. Human glioma tumor cells were plated onto white walled 96 well tissue culture plates at a density of 30,000 cells per mL and allowed to attach overnight. The following day the cells were transfected with scrambled siRNA duplex oligo (Dharmacon Inc.), or with PTPL1/FAP-1 siRNA (SEQ ID NO:3 MGUAAGCCUAGCUGAUCCUG) using Oligofectamine transfection reagent (Invitrogen) according to manufacturers recommendations. The media was changed and cell growth was measured each day for 3 subsequent days. Cell Titer Glo reagent (Promega) was used to measure the growth of these cells at each time point. Protein extracts were made from the cells at each time point and used for immunoblot analysis to measure PTPL1/FAP-1 protein levels. PTPL1/FAP-1 siRNA transfected cells exhibit a significant and consistent inhibition of cell proliferation. These data indicate that PTPL1/FAP-1 is involved in glioma cell growth. By specifically knocking down PTPL1/FAP-1 protein levels in the cancer cells we demonstrate a critical function for this protein in cell growth. These observations would predict that a small molecule inhibitor of PTPL1/FAP-1 function would similarly prevent tumor cell growth.

Apoptosis. The role PTPL1/FAP-1 has in FAS induced apoptosis of human glioma tumor cells was evaluated by cell viability measurements. Human glioma tumor cells were plated onto white walled 96 well tissue culture plates at a density of 30,000 cells per mL and allowed to attach overnight. The following day the cells were transfected with scrambled siRNA duplex oligo (Dharmacon Inc.), or with PTPL1/FAP-1 siRNA (MGUMGCCUAGCUGAUCCUG) using Oligofectamine transfection reagent (Invitrogen) according to manufacturer's recommendations. The media was changed and on the following day the cells were treated with recombinant FAS Ligand (Oncogene Inc.) for 18 hours. Cell viability was measured using Cell Titer Glo reagent (Promega). PTPL1/FAP-1 siRNA transfected cells exhibit a significant increase in sensitivity to FAS induced apoptosis. These data indicate that PTPL1/FAP-1 normally acts to inhibit FAS induced apoptosis and that by specifically knocking down PTPL1/FAP-1, tumor cells become extra sensitive to FAS induced apoptosis.

Time-Resolved Fluorescence Resonance Energy Transfer (TR-FRET) Assay. In order to screen agents and identify modulators of PTPL1/FAP-1, a TR-FRET assay is employed. To each well of a 384 assay plate (Corning 384-well, black) 15 ul of reaction buffer (50 mM HEPES @ pH=8, 1 mM MgCl₂ 2 mM EDTA, 0.01% Brij solution, 1 mM DTT) is added. Into each test well 2.5 μL 0.1 to 0.3 mM compound (dissolved in DMSO) and 12.5 μL 3.5 nM PTPL1/FAP-1 enzyme solution is combined and incubated for 10 minutes at room temperature. 10 μL substrate (AGY1336, or FASpTyr275 peptide; SEQ ID NO:4 Biotin-KKEAY(PO3)DTLI-COOH) solution is added to each well and the plates are mixed by shaking for 2 minutes, followed by a 27° C. incubation for 45 minutes. The positive control wells receive reaction buffer, enzyme and substrate and are treated the same as the test wells. For detection 20 μL detection reagent (1.2 ng Eu-labeled PY20 Antibody, 1 ug/ml Streptavidin-APC, 36 mM SodiumOrthoVanadate) is dispensed to each well. The plates are read for Europium and APC via fluorescence using the excitation wavelength of 337 nm and the emission wavelengths of 615 nm and 665 nm.

Cell Surface expression of FAS assay: Modulators of PTPL1/FAP-1 are tested for upregulation of FAS cell surface expression by the following assay. Human glioma cells are plated onto 96 well plates at 5,000 cells/well. Compounds are added at 10 uM, 1% DMSO final concentration and no compounds controls. The cells are washed and fixed in formalin, blocked and incubated with FAS-ECD specific MAb (and Hoecsht for nuclei) (about 3 hr procedure). Plates were read in Cellomics HCS ArrayScan. Define mask, exposure and threshold for analysis of FAS surface expression (intensity units).

### EXAMPLE 2

### Interaction of FAP-1 and FAS

Materials: Human glioma D566 cells were cultured in MEM Zn⁺ option (Gibco) supplemented with 10% fetal bovine serum (Gibco). U87-MG (ATCC Cell line) cells were cultured in Dulbecco's modified Eagle's medium supplemented with 10% fetal bovine serum (Gibco), 1 mmol/L L-glutamine, 100 U/ml penicillin/streptomycin. All cell line were incubated at 37° C., 5% CO₂.

Quantitative Real Time PCR. Total RNA from normal brain tissue samples and GBM tumor samples was isolated with Trizol (Gibco BRL) according to the manufacturer's instructions. The RT-PCR protocol for the synthesis of cDNA was described previously. SYBR Green real-time PCR amplifications were performed in an iCycler Real-Time Detection System (Bio-Rad Laboratories, Hercules, Calif.). The reactions were carried out in a 96-well plate in a 25-μl reaction volume containing 12.5 μl of 2× SYBR Green Master Mix (PE Applied Biosystems), a 0.9 μM concentration of each forward and reverse primer, 200 ng of total cDNA and supplemented to 25 μl with nuclease-free H₂O (Promega). The thermal profile for all SYBR Green PCRs was 50° C. for 2 minutes and 95° C. for 10 minutes, followed by 45 cycles of 95° C. for 15 seconds, 60° C. for 30 seconds followed by 72° C. for 40 seconds. Quantification was performed using the comparative cycle threshold (CT) method, where CT is defined as the cycle number at which fluorescence reaches a set threshold value. The target transcript was normalized to an endogenous reference (simultaneous triplicate β-actin reactions), and relative differences were calculated using the PCR efficiencies.

Immunohistochemistry: Tumor tissue micro-array slides (Ambion) were placed on a heat block (45° C.) for 4-6 hours, dewaxed and treated with target retrieval solution (Innogenex). The slides were then stained with anti-FAP-1 antibody (Santa Cruz Biotechnology) using an anti-mouse DAB chromagen detection kit according to supplier's instructions (Innogenex).

Immunoprecipitation and Immunoblot Analysis: Cells were lysed in RIPA buffer (0.1% SDS, 1% NP40, and 0.5% sodium deoxycholate in PBS) for 20 minutes on ice and debri cleared by centrifugation. Protein quantification was performed using the BCA method (Pierce). Total cellular proteins were separated on standard 8% SDS-polyacrylamide gels (Invitrogene). The fractionated proteins were transferred onto Nitrocellulose membranes (VWR), blocked in 5% non-fat milk followed by incubation with the indicated anibody: anti-FAP-1 (Santa Cruz Biotechnology), Fas/CD95/APO-1 (BD Transduction Laboratories) and PY20 (Santa Cruz Biotechnology). Immunodetection was accomplished by incubation with horseradish peroxidase-conjugated secondary antibody (Santa Cruz Biotechnology), and enhanced chemiluminescence methods (Amersham), followed by exposure to X-ray film (Kodak). For immunoprecipitation experiments 1 mg of cell lysate in RIPA buffer was incubated with either 5 μg of FAS antibody or 5 μg of FAP-1 antibody and incubated with gentle rocking for 2 hours at 4° C. prior to precipitation of antibody-target complex with Protein A/G agarose (Santa Cruz Biotechnology) for an additional 2 hours. The immunoprecipitation complex was washed three times in RIPA and resuspended in gel loading dye prior to immunoblot analysis with the indicated antibodies.

RNA interference: Human D566 cells were plated in 100 μl at a density of 50,000 cell/ml onto a 96 well plate. Cells were then transfected with 200 nM of FAP-1 specific siRNA: SEQ ID NO:3 GUAAGCCUAGCUGAUCCUG dTdTdTdT CAUUCGGAUCGACUAGGAC (Dharmacon) using Oligofectamine™ Reagent (Invitrogen) according to the suppliers instructions.

Cell viability measurements: Following indicated treatment, cells were assayed for viability using a homogenous mix and read assay reagent according to supplier's instructions (Cell Titer Glo Promega). Luminescence corresponding to the levels of ATP in viable cells was detected using a luminescence plate reader.

Tyrosine Phosphatase Assay: To measure tyrosine phosphatase activity FAP-1 was immunoprecipitated from D566 cells and incubated with the biotinylated phosphopeptide, FASpTyr275 at 10 μg/ml for 30 minutes 37° C. The peptide was then added to NeutrAvidin-coated 96 well plates (Pierce) and incubated for 30 minutes at 37° C. The anti-phosphotyrosine antibody, PY20, conjugated with horseradish peroxidase (HRP) was added for the specific detection of phosphotyrosine peptide. Antibody binding was measured by the addition of the colorimetric HRP-substrate, TMB (Sigma).

Results

Expression profile of FAP-1. To determine the expression profile of FAP-1 mRNA, glioblastoma tumors and normal control tissues were analyzed using real time quantitative PCR. These studies demonstrate that FAP-1 mRNA is expressed at levels in glioblastoma tumor tissue up to 20 times higher then in normal brain tissue. The house keeping gene p-actin was used to normalize the relative abundance of FAP-1 in these surgically removed, flash frozen tumor specimens. Therefore FAP-1 expression is a good diagnostic marker of glioblastoma and a specific target for glioblastoma therapies.

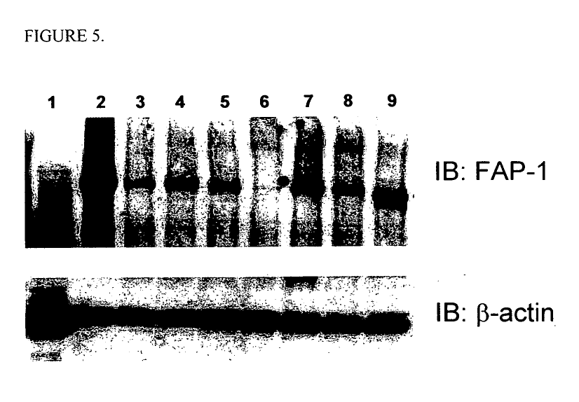

Because mRNA levels may not reflect the magnitude of protein expression, we tested a collection of normal and brain tumor tissue for the relative level of FAP-1 protein by immunohistochemistry (IHC) on paraffin embedded tissue micro-array sections (Tables 1 and 2, FIG. 5). In this study 20 out of 31 glioblastoma tumors (64.5%) over-express FAP-1. In addition astrocytoma grade II and III also display moderate levels of regional FAP-1 up regulation. FAP-1 expression is less robust in Astrocytoma Grades II and III and is more prevalent and intensely expressed in glioblastoma tissue specimens. FAP-1 is found at low to undetectable levels in normal adult human brain and most other human tissues.

TABLE 1
IHC DATA FOR FAP-1 EXPRESSION IN ASTROCYTOMAS

| Histology/Tumor Type | Positive Tumors |
|---|---|
| Normal Cerebellum | 0 of 4 |
| Glioblastoma/Astrocytoma IV | 20 of 31 |
| Astrocytoma III | 2 of 4 |
| Astrocytoma II | 2 of 4 |

An examination of a panel of human cancer tissues demonstrate that FAP-1 is up regulated in several adenocarcinomas, specifically, breast, ovarian, colon, and prostate tumor samples (Table 2). In breast and prostate tumors FAP-1 protein is over expressed when compared to several normal or matched benign specimens for each of these tumor types. These data demonstrate that FAP-1 expression is a good diagnostic marker of certain cancers, and a specific target for therapy.

TABLE 3
IHC DATA FOR FAP-1 EXPRESSION IN CANCER

| Cancer Tissue | Histology | Positive Tumors | Normal Tissue |
|---|---|---|---|
| Breast | Adenocarcinoma | 4 of 15 (27%) | 0 of 7 (0%) |
| Ovary | Cystadenocarcinoma | 6 of 10 (60%) | 0 of 2 (0%) |
| Endometrium | Adenocarcinoma | 3 of 7 (42%) | ND |
| Stomach | Adenocarcinoma | 5 of 5 (100%) | 0 of 1 (0%) |
| Colon | Adenocarcinoma | 11 of 12 (92%) | 1 of 3 (33%) |
| Pancreas | Adenocarcinoma | 1 of 9 (11%) | 1 of 6 (17%) |
| Liver | Hepatocarcinoma | 4 of 6 (67%) | 2 of 4 (50%) |
| Renal/Pelvis | Transitional | 2 of 8 (25%) | ND |
| | Carcinoma | | |
| Kidney | Renal Carcinoma | 11 of 15 (73%) | 4 of 6 (67%) |
| Bladder | Transitional | 16 of 20 (80%) | 0 of 1 (0%) |
| | Carcinoma | | |
| Prostate | Adenocarcinoma | 7 of 13 (54%) | 0 of 8 (0%) |
| Skin | Melanoma | 4 of 5 (80%) | 0 of 2 (0%) |
| Esophagous | Adenocarcinoma | 5 of 6 (83%) | 0 of 1 (0%) |
| Lip/Tongue/ | Squamous | 24 of 28 (85%) | 2 of 6 (33%) |
| Mouth | | | |
| Paratoid | Mixed Tumor | 5 of 7 (71%) | ND |
| Larynx | Squamous | 5 of 5 (100%) | 1 of 2 (50%) |
| Pharynx | Squamous | 6 of 6 (100%) | 0 of 1 (0%) |
| Lymph Node | Lymphoma | 3 of 7 (42%) | 0 of 2 (0%) |
| Lung | Squamous/Adeno. | 2 of 9 (22%) | 1 of 3 (33%) |

To complete the expression profiling studies of FAP-1 we examined a panel of cell lines to determine the relative levels of expression (FIG. 5). Jurkat cells act as a negative control and HEK 293 cells were used as a positive expressing cell line. Examination of several human glioma cell lines demonstrate variable levels of FAP-1 expression. D566 cells were used in subsequent studies to characterize FAP-1 function based on their intermediate to high levels of FAP-1 protein expression.

FAP-1 siRNA decreases glioma cell viability. By specifically knocking down FAP-1 protein levels in glioma cells we demonstrate a critical function for this protein in cell viability. FAP-1 siRNA transfected cells exhibit a significant decrease in cell viability. In contrast, a scrambled control siRNA did not decrease cell viability. Data is shown in Flgure 3. Consistent with these observations, a decrease in FAP-1 protein levels is observed in cells transfected with FAP-1 siRNA. This effect is time dependent and peaks on day 3. In contrast the loading control protein β-actin did not change, demonstrating a FAP-1 specific knock-down.

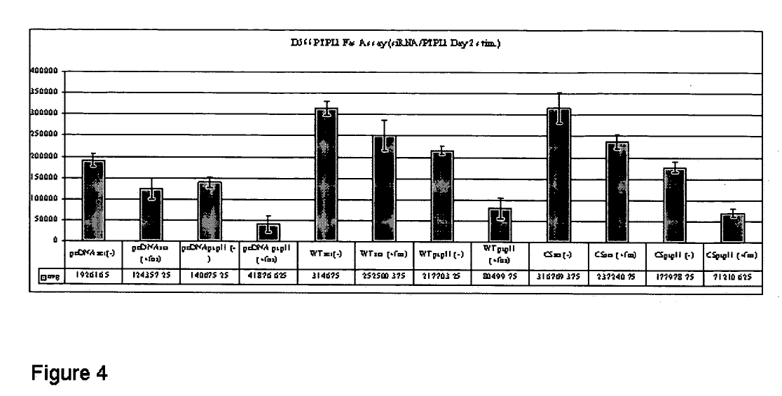

To test the role of FAP-1 in cell survival we studied the response of glioblastoma cells to FASL mediated apoptosis. Resistance to apoptosis is an important aspect of glioblastoma biology. FAP-1 siRNA transfected cells exhibit a significant increase in FASL induced apoptosis. These data, shown in FIG. 4, indicate that FAP-1 siRNA can be used to enhance FASL induced apoptosis. Therefore, by specifically knocking down or inhibiting FAP-1, the glioblastoma cells become extra sensitive to FASL induced apoptosis.

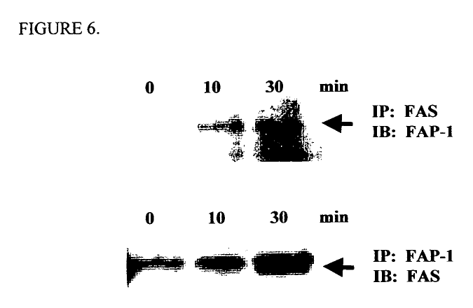

Functional interaction between FAP-1 and FAS. In order to determine the mechanism of action by which FAP-1 enhances FASL induced cell death, we tested for the physical association between FAP-1 and FAS. In co-immunoprecipitation experiments, FAS antibodies were used to pull down FAP-1, and conversely FAP-1 antibodies were used to pull down FAS (FIG. 6). The D566 glioma cells were treated with FASL for the indicated times prior to cell lysis and immunoprecipitation. Cells treated with FASL demonstrate an inducible association between FAP-1 and FAS. Some constitutive association was observed in untreated cells.

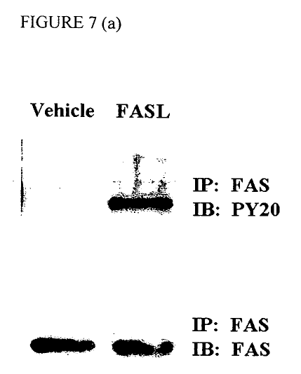

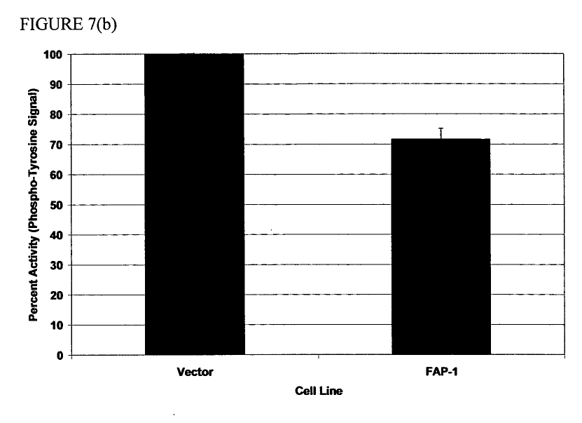

The physical association between FAP-1 and FAS indicates that FAP-1 is a direct modulator of FAS activity and may influence its tyrosine phosphorylation status. Human FAS contains a consensus tyrosine phosphorylation site at tyrosine residue 275. Indeed, treatment of D566 glioma cells with FASL induced the tyrosine phosphorylation of FAS. In this experiment, human D566 glioma cells were treated with FASL for 30 minutes and FAS was immunoprecipitated and blotted with anti-phosphotyrosine antibodies (FIG. 6a). We next tested if FAP-1 had tyrosine phosphatase activity towards a phospho-tyrosine FAS peptide substrate (FASpTyr275). We used an ELISA based in vitro immunoprecipitate-complex tyrosine phosphatase procedure to measure FAP-1 activity. The FASpTyr275 biotinylated peptide was incubated with FAP-1 immunoprecipitate for 30 minutes and then the level of dephosphorylated peptide was measured relative to vector control by an ELISA based anti-phosphotyrosine procedure. Together these results indicate that FAP-1 directly binds and dephosphorylates FAS following stimulation with FASL (FIG. 7).

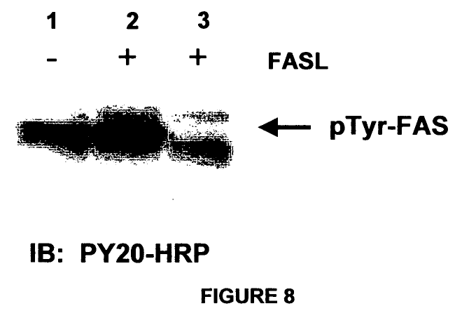

The phosphorylation of FAS in response to FASL; and the dephosphorylation of FAS by FAP-1 is shown by the following experiment. The data is provided in FIG. 8. Human glioma cells (D566) were plated at subconfluency and allowed to attach overnight. The next day the media was replaced with Optimem and the cells incubated for 3 hours. FASL or media control was added for 30 minutes. The cells were lysed in eukaryotic lysis buffer. Lane 1: Unstimulated cells immunoprecipitated with FAS and IgG control antibodies. Lane 2: FASL stimulated cells immunoprecipitated with FAS and IgG control antibodies. Lane 3: FASL stimulated cells immunoprecipitated with FAS and FAP-1 antibodies. The immunoprecipitated complexes were washed and incubated at 30 C for 30 minutes in phosphatase reaction buffer. Loading dye was added to the samples to stop the reaction and the gels ran. The blot was probed using anti-phosphotyrosine antibody conjugated to horse radish peroxidase (PY20-HRP). The arrow indicates tyrosine phosphorylated FAS (pTyr-FAS).

Several PTPs have been studied for their role in cancer. By dephosphorylating target tyrosines, PTPs can modulate the activity of effector proteins. The extensive number of PTP family members suggests that their activities are selective and drugs directed at phosphatases would modulate a specific biochemical pathway. Therefore, if one could link a particular PTP activity to a disease related pathway then the developed drug would be targeted. In contrast, most chemotherapy agents are largely non-specific to the particular cancer. This is especially relevant to the treatment of GBM, a tumor that is extremely resistant to classical chemotherapy and radiation therapies.

Glioblastomas undergo apoptosis through p53 dependent and independent pathways. Modulating the FAS pathway is one such way that gliomas can be made sensitive to apoptosis regardless of their p53 status. FAP-1 is a key regulator of FAS and controls both its cell surface expression and its signaling capacity. The activities of FAP-1 require its ability to directly interact with FAS.

The above results demonstrate that FASL induces association between FAP-1 and FAS. Further, by knocking down the expression of FAP-1 or mutating/deleting certain domains, the trafficking of FAS to the tumor cell surface is increased. This effect is thought to be mediated largely by protein-protein interactions between FAP-1 and FAS, which requires binding with either the third or fifth PDZ domain of FAP-1 with the C-terminus of FAS. Disrupting the interaction between FAP-1 and FAS with the SLV FAS C-term tri-peptide results in increased sensitivity of tumor cells to FASL induced apoptosis. It is demonstrated hererin that by using RNAi mediated FAP-1 knockdown, followed by treatment of glioma cells with FASL, cell death is increased.

Cells transfected with a FAP-1 phosphatase mutant are more sensitive to FASL than FAP-1 phosphatase competent transfectants indicating that FAP-1 phosphatase activity is involved in FASL mediated apoptosis. The above results demonstrate that FAP-1 dephosphorylates FAS at tyrosine 275 and thereby downregulates FASL mediated apoptosis. Both the trafficking and phosphatase activities of FAP-1 are important to the regulation of FAS. Small molecules that interfere with FAP-1 phosphatase activity can be used to enhance the sensitivity of cancer cells to FASL mediated apoptosis. Inhibitors of FAP-1 would upregulate FAS surface expression and thereby enhance its pro-apoptotic effects. Identification of such compounds is of great benefit to the treatment of glioblastoma and other cancers.

The foregoing is intended to be illustrative of the embodiments of the present invention, and is not intended to limit the invention in any way. Although the invention has been described with respect to specific modifications, the details thereof are not to be construed as limitations, for it will be apparent that various equivalents, changes and modifications may be resorted to without departing from the spirit and scope thereof and it is understood that such equivalent embodiments are to be included herein. All publications and patent applications are herein incorporated by reference to the same extent as if each individual publication or patent application was specifically and individually indicated to be incorporated by reference.

What is claimed is:

1. A method of screening candidate agents for modulation of a tumor target protein, the method comprising:
combining a candidate biologically active agent with any one of:
(a) a PTPL1/FAP-1 polypeptide;
(b) a cell comprising a nucleic acid encoding and expressing a PTPL1/FAP-1 polypeptide; or
(c) a non-human transgenic animal model for brain tumor gene function comprising one of:
(i) a knockout of a gene encoding PTPL1/FAP-1; (ii) an exogenous and stably transmitted mammalian gene sequence encoding PTPL1/FAP-1; and
determining the effect of said agent on PTPL1/FAP-1, wherein agents that modulate PTPL1/FAP-1 activity provide for molecular and cellular changes in tumor cells.

2. The method according to claim 1, wherein said biologically active agent downregulates expression.

3. The method according to claim 1, wherein said biologically active agent inhibits activity of said polypeptide.

4. A method for the diagnosis or staging of a brain tumor, the method comprising:
detecting the level of PTPL1/FAP-1 in brain cells;
comparing said level of PTPL1/FAP-1 to a normal sample of comparable cells;
wherein an increase in PTPL1/FAP-1 is indicative that that cells are tumor cells.

5. The method according to claim 4, wherein said brain tumor is an astrocytoma.

6. The method according to claim 5, wherein said astrocytoma is a glioblastoma.

7. The method according to claim 4, wherein said detecting comprises detecting increased amounts of mRNA in said cells.

8. The method according to claim 4, wherein said detecting comprises detecting increased amounts of PTPL1/FAP-1 polypeptide.

9. The method according to claim 4, wherein said detecting comprises detecting increased levels of PTPL1/FAP-1 enzymatic activity.

10. A method to treat a brain tumor, the method comprising:
administering a therapeutic amount of a compound identified by the method of claim 1.

11. The method of claim 10 wherein said compound is administered by intrathecal administration.

12. The method of claim 10 wherein said compound is administered by intravascular administration.

13. The method of claim 10, wherein said compound is administered by oral administration.

14. The method of claim 10 wherein the brain tumor is an astrocytoma.

15. The method of claim 14, wherein said astrocytoma is a glioblastoma.

16. The method of claim 10, further comprising administering a therapeutic amount of a second agent.

17. The method of claim 16, wherein said second agent is a chemosensitizing agent.

18. The method of claim 16, wherein said second agent is a radiation sensitizing agent.

19. A method for the diagnosis or staging of a tumor, the method comprising:
detecting the level of PTPL1/FAP-1 in tumor cells;
comparing said level of PTPL1/FAP-1 to a normal sample of comparable cells;
wherein an increase in PTPL1/FAP-1 is indicative that that cells are tumor cells.

20. The method according to claim 19, wherein said detecting step comprises detecting the phosphatase activity of PTPL1/FAP-1 on a FAS substrate.

21. A method to treat a tumor, the method comprising:
administering a therapeutic amount of a compound identified by the method of claim 1.

22. The method according to claim 21, wherein said tumor is selected from the group consisting of ovary cystadenocarcinoma; endometrium adenocarcinoma; stomach adenocarcinoma; liver hepatocarcinoma; renal/pelvis transitional carcinoma; kidney renal carcinoma; bladder transitional carcinoma; prostate adenocarcinoma; skin melanoma; esophagous adenocarcinoma; mouth squamous carcinoma; paratoid mixed tumor; larynx squamous carcinoma; pharynx squamous carcinoma; lymph node lymphoma; lung squamous/adenocarcinoma.

23. The method of claim 22, further comprising administering a therapeutic amount of a second agent.

24. The method of claim 23, wherein said second agent is a chemosensitizing agent.

25. The method of claim 23, wherein said second agent is a radiation sensitizing agent.
